(19) United States
(12) Patent Application Publication
(10) Pub. No.: US 2025/0017874 A1
(43) Pub. Date: Jan. 16, 2025

(54) PHARMACEUTICAL COMPOSITIONS FOR USE IN REDUCING HYPERPIGMENTATION

(71) Applicant: Caliway Biopharmaceuticals Co., Ltd., New Taipei City (TW)

(72) Inventor: Yu-Fang LING, New Taipei City (TW)

(21) Appl. No.: 18/762,851

(22) Filed: Jul. 3, 2024

Related U.S. Application Data
(60) Provisional application No. 63/512,421, filed on Jul. 7, 2023.

(51) Int. Cl. A61K 31/05; A61K 9/00; A61K 45/06; A61K 47/14; A61K 47/44; A61P 17/00

(52) U.S. Cl. CPC A61K 31/05; A61K 9/0014; A61K 45/06; A61K 47/14; A61K 47/44; A61P 17/00

(57) ABSTRACT

A method for reducing hyperpigmentation in a subject, comprising administering an effective amount of a pharmaceutical composition to a skin site of a subject in need thereof, wherein the pharmaceutical composition comprises a pharmaceutically acceptable carrier and a stilbene compound, for example, a compound of Formula (I) (e.g., Formula (I-A)), or a pharmaceutically acceptable salt thereof.

### CROSS REFERENCE TO RELATED APPLICATIONS

This application claims the benefit of the filing date of U.S. Provisional Application No. 63/512,421, filed Jul. 7, 2023, the entire contents of which are incorporated by reference herein.

### BACKGROUND

Hyperpigmentation causes patches of skin to become darker than the surrounding skin. It occurs when the skin produces excess melanin. Pigmentation of the skin results from the accumulation of melanin-containing melanosomes in the basal layer of the epidermis.

Melanins are polymorphous and multifunctional biopolymers which are represented by eumelanin, pheomelanin and neuromelanin. An appropriate amount of melanin in the skin can absorb ultraviolet (UV) lights and maintain the skin's body temperature, thereby protecting the skin from UV light. However, Excessive melanin can make the skin look dull, resulting in an unpleasant appearance. Further, the excess of melanin can lead to pigmented skin diseases such as skin melanization or spots and freckles, etc. Melanin production can also cause problems in other organs, e.g., animal skin, hair, eyes, ears, or brain.

It is therefore of interest to develop compositions capable of modulating melanin levels, thereby reducing hyperpigmentation. Therefore, the ability to remove or lessen the appearance of hyperpigmentation (e.g., melasma, seborrheic keratosis, chloasma, freckles, sunburn, or aging skin) can be of interest to individuals desiring a uniform skin color and a more radiant complexion. Report on “2017-2022 Monitoring and Prospect Forecast of Chinese Whitening Market” issued by the Intelligence Research Group indicates that more than 80% of Chinese females have a demand for skin whitening for those between the ages of 20 to 50, and there is a nearly 66 billion whitening product market every year. Therefore, there is a significant market for a safe and effective product for controlling skin pigmentation.

### SUMMARY

The present application is based, at least in part, on the surprising discovery that a stilbene-based compound (e.g., 4-[(E)-2-(3,5-dihydroxyphenyl)ethenyl]benzene-1,3-diol) exhibited significant melanin-inhibiting activity, both in vitro and in vivo. Accordingly, such compounds are expected to be highly effective in reducing hyperpigmentation.

Accordingly, provided herein is a method for reducing hyperpigmentation in a subject, comprising administering an effective amount of a pharmaceutical composition to a skin site of a subject in need thereof.

In some embodiments, the pharmaceutical composition comprises a pharmaceutically acceptable carrier and a stilbene compound, which may have the structure of Formula (I) or a pharmaceutically acceptable salt thereof:

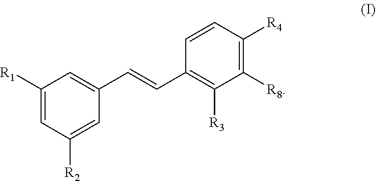

in which each of R₁, R₂, R₃, R₄, and R₈ independently, is H, halogen, thiol, hydroxyl, C₁₋₄ alkyl, C₂₋₄ alkenyl, OR₅, COOR₆, or —OC(═O)R₇. Each of R₅, R₆, and R₇ are independently H, or C1-4 alkyl.

In some examples, the stilbene compound disclosed herein may have the structural of Formula (I-A):

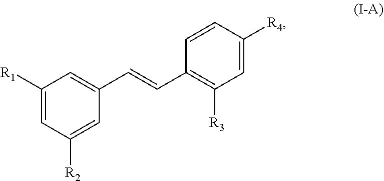

in which each of R₁, R₂, R₃, and R₄ is as defined herein.

In some instances, at least one of R₁, R₂, R₃, and R₄ is hydroxyl. For example, three of R₁, R₂, R₃, and R₄ in the Formula (I) or Formula (I-A) compounds provided herein may be hydroxyl. In some instances, the other one can be hydrogen. In other examples, all of R₁, R₂, R₃, and R₄ may hydroxyl.

In some instances, at least one of R₁, R₂, R₃, and R₄ is methoxy. For example, three of R₁, R₂, R₃, and R₄ in the Formula (I) or Formula (I-A) compounds provided herein may be methoxy. In some instances, the other one can be hydrogen. In other examples, all of R₁, R₂, R₃, and R₄ may methoxy.

In some instances, at least one of R₁, R₂, R₃, and R₄ is OC(═O)R₇, in which R₇ is C₁0.4 alkyl (e.g., methyl). For example, three of R₁, R₂, R₃, and R₄ in the Formula (I) or Formula (I-A) compounds provided herein may be OC(═O)R₇, in which R₇ is C₁0.4 alkyl (e.g., methyl). In some instances, the other one can be hydrogen. In other examples, all of R₁, R₂, R₃, and R₄ OC(═O)R₇, in which R₇ is C₁0.4 alkyl (e.g., methyl).

Any of the Formula (I) compounds provided herein may hydroxyl at position R₈.

Exemplary stilbene compounds are provided below:

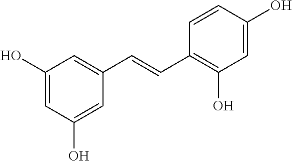 [SMILES: Oc1cc(O)cc(/C=C/c2ccc(O)cc2O)c1]

(4-[(E)-2-(3,5-dihydroxyphenyl)ethenyl]benzene-1,3-diol),

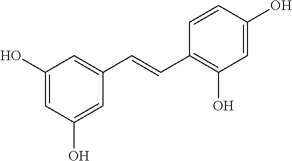 [SMILES: Oc1cc(O)cc(/C=C/c2ccc(O)cc2O)c1]

(5-[(E)-2-(4-hydroxyphenyl)ethenyl]benzene-1,3-diol),

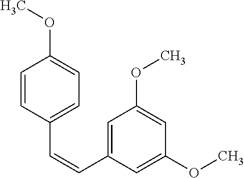

(1,2,3-trimethoxy-5-[(Z)-2-phenylethenyl]benzene),

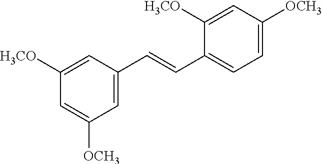 [SMILES: COc1cc(/C=C/c2ccc(OC)cc2OC)cc(OC)c1]

(1-[(E)-2-(2,4-dimethoxyphenyl)ethenyl]-3,5-dimethoxybenzene),

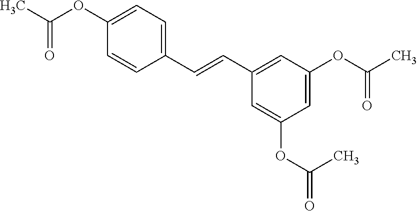 [SMILES: CC(=O)Oc1ccc(/C=C/c2cc(OC(C)=O)cc(OC(C)=O)c2)cc1]

([4-[(E)-2-(3,5-diacetyloxyphenyl)ethenyl]phenyl]acetate), and

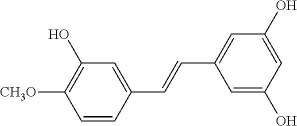

(5-[(E)-2-(3-hydroxy-4-methoxyphenyl)ethenyl]benzene-1,3-diol).

In some examples, the stilbene compound is not resveratrol.

In some embodiments, the pharmaceutically acceptable carrier used in any of the pharmaceutical compositions described herein may comprise a non-ionic surfactant having a hydrophilic-lipophilic balance value (HLB value) greater than 5, a solvent (e.g., a polyol or a polymer thereof such as ethylene glycol, polyoxyethylene, polyethylene glycol, or propylene glycol), acetone, dimethyl sulfoxide, glycerol, alcohol, or a combination thereof), a buffer agent, an oil phase (e.g., soybean oil and/or medium-chain triglycerides), or a combination thereof. In some specific examples, the non-ionic surfactant comprises polyoxyl castor oil (e.g., polyoxyl 35 castor oil), and/or polyoxyl hydrogenated castor oil (e.g., polyoxyl 40 hydrogenated castor oil).

In some embodiments, the pharmaceutical composition may comprise a plurality of nanoparticles formed by the pharmaceutically acceptable carrier and the stilbene compound such as a Formula (A), or a pharmaceutically acceptable salt thereof. In some examples, the nanoparticles have an average particle size of up to 200 nm and a polydispersity index (PDI) value of up to 0.4. In some instances, the pharmaceutically acceptable carrier comprises any of the non-ionic surfactants disclosed herein. Alternatively or in addition, the weight ratio between the compound of Formula (A) or the pharmaceutically acceptable salt thereof to the non-ionic surfactant ranges from 1:1 to 1:1000. In specific examples, the weight ratio ranges from 1:1 to 1:500.

In some embodiments, the pharmaceutical composition described herein may further comprise an additional active agent for reducing hyperpigmentation, which is different from the stilbene compound such as the compound of Formula (A) or the pharmaceutically acceptable salt thereof. In other embodiments, the stilbene compound such as the compound of Formula (A) or the pharmaceutically acceptable salt is the sole active agent in the pharmaceutical composition for reducing hyperpigmentation.

In some embodiments, the method described herein may further comprise administering to the subject an additional active agent for reducing hyperpigmentation, which is different from the stilbene compound such as the compound of Formula (A) or the pharmaceutically acceptable salt thereof. In other embodiments, the method is free from giving the subject such an additional active agent.

In some instances, the pharmaceutical composition may further comprise an antioxidant. Alternatively, the pharmaceutical composition is free of any oxidant.

In some embodiments, the pharmaceutical composition provided herein is free of water.

Any of the pharmaceutical compositions disclosed herein can be administered to the skin site via intradermal injection or topical application.

Also provided herein is any of the pharmaceutical compositions disclosed herein for use in reducing hyperpigmentation in a subject, as well as use of such a pharmaceutical composition for manufacturing a medicament for use in reducing hyperpigmentation in a subject.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a diagram showing the changes of melanin production in mouse melanoma B16-F10 cells treated with various 4-[(E)-2-(3,5-dihydroxyphenyl)ethenyl]benzene-1,3-diol formulations.

FIG. 2 is a bar graph showing inhibitory activity against melanin production by the various 4-[(E)-2-(3,5-dihydroxyphenyl)ethenyl]benzene-1,3-diol formulations on mouse melanoma B16-F10 cells.

FIG. 3 is a photo showing darkness of mouse back skins after UVB irradiation and treatment by the compositions as indicated. N=3-4/group. Data was analyzed by Student's t-test. #p<0.05, ##p<0.01 compared with Control; *p<0.05, **p<0.01 compared with UVB.

FIGS. 4A-4C include graphs showing reduction of melanin in mice treated with 4-[(E)-2-(3,5-dihydroxyphenyl)ethenyl]benzene-1,3-diol compositions. FIG. 4A is a graph showing the reduction melanin content in mice skin after UVB irradiation and treatment by exemplary 4-[(E)-2-(3,5-dihydroxyphenyl)ethenyl]benzene-1,3-diol compositions as indicated. N=3˜4/group. Data was analyzed by Student's t-test. #p<0.05, ##p<0.01 compared with Control; *p<0.05, **p<0.01 compared with UVB. FIG. 4B is a graph showing the reduction of melanin content in mice skin after UVB irradiation and treatment by the exemplary 4-[(E)-2-(3,5-dihydroxyphenyl)ethenyl]benzene-1,3-diol composition as indicated via intradermal injection. N=4-8/group. Data was analyzed by Student's t-test. #p<0.05, ##p<0.01, ###p<0.001 compared with Control; *p<0.05, **p<0.01 compared with UVB. FIG. 4C is a graph showing the reduction of melanin content in mice skin after UVB irradiation and treatment by the exemplary 4-[(E)-2-(3,5-dihydroxyphenyl)ethenyl]benzene-1,3-diol composition as indicated via topical administration. N=4-8/group. Data was analyzed by Student's t-test. #p<0.05, ##p<0.01, ###p<0.001 compared with Control; *p<0.05, **p<0.01 compared with UVB.

FIG. 5 is a graph showing the changes of melanin production in mouse melanoma B16-F10 cells treated with 4-[(E)-2-(3,5-dihydroxyphenyl)ethenyl]benzene-1,3-diol versus 5-[(E)-2-(4-hydroxyphenyl)ethenyl]benzene-1,3-diol and other whitening ingredients as indicated.

FIGS. 6A-6C include diagraphs showing the impact of 4-[(E)-2-(3,5-dihydroxyphenyl)ethenyl]benzene-1,3-diol on α-MSH-induced melanin production as compared with other ingredients such as arbutin and hydroquinone. FIG. 6A: a graph showing that α-MSH induces melanin production. FIG. 6B: a graph showing the changes of melanin production in mouse melanoma B16-F10 cells treated with α-MSH and various 4-[(E)-2-(3,5-dihydroxyphenyl)ethenyl]benzene-1,3-diol compositions and arbutin and hydroquinone compositions as indicated. FIG. 6C: a graph showing the changes of melanin production in mouse melanoma B16-F10 cells treated with α-MSH and various 4-[(E)-2-(3,5-dihydroxyphenyl)ethenyl]benzene-1,3-diol compositions and arbutin compositions as indicated.

FIGS. 7A-7D include diagraphs showing in vitro melanin reduction activity and cytotoxicity of 4-[(E)-2-(3,5-dihydroxyphenyl)ethenyl]benzene-1,3-diol, arbutin, and hydroquinone. FIG. 7A: melanin levels in cells induced by α-MSH and treated with 4-[(E)-2-(3,5-dihydroxyphenyl)ethenyl]benzene-1,3-diol at different concentrations. FIG. 7B: viability of cells treated with 4-[(E)-2-(3,5-dihydroxyphenyl)ethenyl]benzene-1,3-diol at different concentrations. FIG. 7C: melanin levels in cells induced by α-MSH and treated with 4-[(E)-2-(3,5-dihydroxyphenyl)ethenyl]benzene-1,3-diol, arbutin, or hydroquinone. FIG. 7D: viability of cells treated with 4-[(E)-2-(3,5-dihydroxyphenyl)ethenyl]benzene-1,3-diol, arbutin, or hydroquinone. N=3/group; N.D.=Not detected. Data was analyzed by Student's t-test. #p<0.05, ##p<0.01, ###p<0.001 compared with Vehicle; *p<0.05, **p<0.01, ***p<0.01 compared with α-MSH.

### DETAILED DESCRIPTION

5-[(E)-2-(4-hydroxyphenyl)ethenyl]benzene-1,3-diol and derivatives thereof such as 4-[(E)-2-(3,5-dihydroxyphenyl)ethenyl]benzene-1,3-diol are polyphenol compounds, which may be found in various natural sources, for example, in berries and skin of grapes. 4-[(E)-2-(3,5-dihydroxyphenyl)ethenyl]benzene-1,3-diol, also known as E-2,3′,4,5′-tetrahydroxystilbene, is a poly hydroxystilbene compound having the structure of

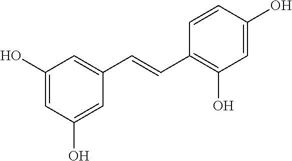 [SMILES: Oc1cc(O)cc(/C=C/c2ccc(O)cc2O)c1]

Various bioactivities have been reported for 5-[(E)-2-(4-hydroxyphenyl)ethenyl]benzene-1,3-diol derivatives such as 4-[(E)-2-(3,5-dihydroxyphenyl)ethenyl]benzene-1,3-diol, including anti-tumor and neuroprotective activities.

Melanin is a group of natural pigments found in most organisms. The melanin pigments are produced in a specialized group of cells known as melanocytes. There are five basic types of melanin: eumelanin, pheomelanin, neuromelanin, allomelanin and pyomelanin. The most common type is eumelanin, of which there are two types brown eumelanin and black eumelanin. Eumelanin is produced through a multistage chemical process known as melanogenesis, where the oxidation of the amino acid tyrosine is followed by polymerization.

Pigmentation of the skin results from the accumulation of melanin-containing melanosomes in the basal layer of the epidermis. In mammalian skin, melanogenesis is initiated by exposure to UV radiation, causing the skin to darken. Differences in skin pigmentation result both from the relative ratio of eumelanin (brown/black) to pheomelanin (yellow/red), as well as the number of melanosomes within melanocytes.

It is reported herein that 5-[(E)-2-(4-hydroxyphenyl)ethenyl]benzene-1,3-diol and derivatives thereof such as 4-[(E)-2-(3,5-dihydroxyphenyl)ethenyl]benzene-1,3-diol successfully reduced melanin levels in cell cultures and in animal models and that 4-[(E)-2-(3,5-dihydroxyphenyl)ethenyl]benzene-1,3-diol is more effective in reducing melanin levels as compared with known whitening agents arbutin and hydroquinone. Accordingly, 5-[(E)-2-(4-hydroxyphenyl)ethenyl]benzene-1,3-diol and derivatives thereof such as 4-[(E)-2-(3,5-dihydroxyphenyl)ethenyl]benzene-1,3-diol would be expected to be effective in reducing skin hyperpigmentation, which could confer various therapeutic and/or cosmetic effects. For instance, 5-[(E)-2-(4-hydroxyphenyl)ethenyl]benzene-1,3-diol and 4-[(E)-2-(3,5-dihydroxyphenyl)ethenyl]benzene-1,3-diol can reduce oxidative damage, premature skin aging, abnormal or hyper-pigmentation, and other age-related disorders.

Accordingly, the prevent disclosure provides a pharmaceutical composition comprising one or more stilbene compounds for use in reducing skin hyperpigmentation in a subject in need of such treatment.

### Definitions

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Exemplary C₁₋₄ alkyl groups include, but are not limited to, methyl, ethyl, n-propyl, isopropyl, n-butyl, sec-butyl, isobutyl, tert-butyl. Exemplary C₂₋₄ alkenyl groups include, but are not limited to, ethenyl, propenyl, butenyl; alkynyl; aralkyl; halogenated alkyl; heteroalkyl; aryl; heterocyclyl; cycloalkyl; cycloalkenyl; cycloalkynyl.

As used herein, the term “alkyl” refers to a linear, saturated, acyclic, monovalent hydrocarbon radical or branched, saturated, acyclic, monovalent hydrocarbon radical, having from one to three carbon atoms attached to the rest of the molecule by a single bond, e.g., methyl, ethyl, n-propyl, or 1-methylethyl (iso-propyl). An optionally substituted alkyl radical is an alkyl radical that is optionally substituted, valence permitting, by one, two, three, four, or five substituents independently selected from the group consisting of halo, cyano, nitro, oxo, hydroxyl, thio, or amino.

As used herein, the term “alkenyl” refers to a linear, acyclic, monovalent hydrocarbon radical or branched, acyclic, monovalent hydrocarbon radical, containing a carbon-carbon double bond, having two or three carbon atoms attached to the rest of the molecule by a single bond, e.g., ethenyl, or propenyl. An optionally substituted alkenyl radical is an alkenyl radical that is optionally substituted, valence permitting, by one, two, or three substituents independently selected from the group consisting of: halo, cyano, nitro, hydroxyl, thio, or amino. As described herein, an alkene may be a Z or E alkene. An alkene shown as a Z alkene include the E isomer of the alkene.

“Amino” refers to a radical of the formula —NRbRc where Rb and Rc are each hydrogen, or an alkyl radical as defined above containing one to three carbon atoms. The alkyl part of the optionally substituted amino radical is optionally substituted as defined above for an alkyl radical.

“Thiol” refers to a radical of the formula —SRd where Rd is a hydrogen or an alkyl radical as defined above containing one to three carbon atoms. The alkyl part of the optionally substituted thiol radical is optionally substituted as defined above for an alkyl radical.

### I. Stilbene Compounds

In some aspects, provided herein are stilbene compounds for use in reducing hyperpigmentation in a subject. As reported herein, an exemplary stilbene compound, 4-[(E)-2-(3,5-dihydroxyphenyl)ethenyl]benzene-1,3-diol, showed great bioactivity in reducing melanin both in vivo and in vitro with little or no cytotoxicity. The melanin reduction activity achieved by 4-[(E)-2-(3,5-dihydroxyphenyl)ethenyl]benzene-1,3-diol was significantly greater than arbutin and HQ, which are known compounds for use in skin whitening or reducing hyperpigmentation.

As used herein, a stilbene compound refers to a compound having a core structure of:

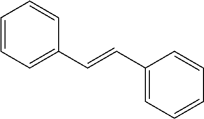 [SMILES: C(=C\c1ccccc1)/c1ccccc1]

with suitable substitutions at one or more positions on either or both of the ring structures. The stilbene compounds for use in any of the methods disclosed herein may include 5-[(E)-2-(4-hydroxyphenyl)ethenyl]benzene-1,3-diol and derivatives thereof, or a pharmaceutically acceptable salt thereof. In some examples, the stilbene compound is a 5-[(E)-2-(4-hydroxyphenyl)ethenyl]benzene-1,3-diol derivative, such as 4-[(E)-2-(3,5-dihydroxyphenyl)ethenyl]benzene-1,3-diol or others provided herein.

In one embodiment, the stilbene compound may have the following Formula (I):

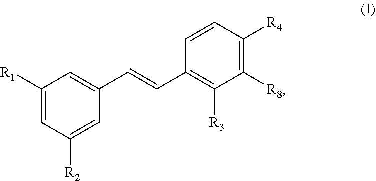

or a pharmaceutically acceptable salt thereof. In Formula (I), each of R₁, R₂, R₃, R₄, and R₈ independently, is H, halogen, thiol, hydroxyl, C₁₋₄ alkyl, C₂₋₄ alkenyl, OR₅, COOR₆, or —OC(═O)R₇, wherein each of R₅, R₆, and R₇ are independently H, or C₁₋₄ alkyl (e.g., methyl).

In some examples, the Formula (I) compounds provided herein may have a structure of Formula (I-A):

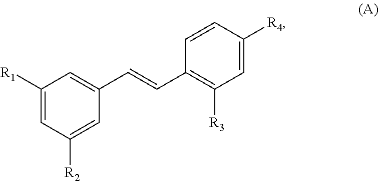

or its pharmaceutically acceptable salt, wherein R₁-R₄ are independently or jointly H, halogen (e.g., Cl or F), thiol, hydroxyl, C₁₋₄ alkyl, C₂₋₄ alkenyl, OR₅, COOR₆, or —OC(═O)R₇, in which each of R₅, R₆, and R₇ are independently H, or C₁0.4 alkyl (e.g., methyl). Alternatively, each of R₁—R₄, independently, can be hydroxyalkyl, thiol, alkoxy, or alkaryl.

In some instances, one or more of R₁-R₄ may be hydroxyl. Alternatively, one or more of R₁-R₄ may be methoxy. In other instances, one or more of R₁-R₄ may be OC(═O)R₇, in which R₇ is H or C₁₋₄ alkyl (e.g., methyl).

Exemplary stilbene compounds are provided herein. In one specific example, the stilbene compound is a 5-[(E)-2-(4-hydroxyphenyl)ethenyl]benzene-1,3-diol derivative, for example, 4-[(E)-2-(3,5-dihydroxyphenyl)ethenyl]benzene-1,3-diol, or a pharmaceutically acceptable salt thereof.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Tnterscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

### II. Pharmaceutical Compositions

Any of the stilbene compounds disclosed here may be mixed with one or more pharmaceutically acceptable carriers to form a pharmaceutical composition, which can be used for reducing hyperpigmentation in a subject who needs the treatment. Pharmaceutically acceptable carriers include diluents, fillers, salts, buffers, stabilizers, solubilizers and other material which are well-known in the art. Exemplary pharmaceutically acceptable carriers in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from a suitable inorganic base, (e.g., sodium hydroxide, barium hydroxide, iron (ii) hydroxide, iron (III) hydroxide, magnesium hydroxide, calcium hydroxide, aluminium hydroxide, ammonium hydroxide, potassium hydroxide, caesium hydroxide, or lithium hydroxide) or a suitable organic base (e.g., pyridine, methyl amine, imidazole, benzimidazole, histidine, phosphazene bases, or a hydroxide of an organic cation such as quaternary ammonium hydroxide and phosphonium hydroxide). Also, pharmaceutically-acceptable salts can be prepared as alkaline metal or alkaline earth salts, such as lithium, sodium, potassium or calcium salts.

The pharmaceutical compositions as described herein can comprise pharmaceutically acceptable carriers, excipients, or stabilizers in the form of lyophilized formulations or aqueous solutions. Remington: The Science and Practice of Pharmacy 20th Ed. (2000) Lippincott Williams and Wilkins, Ed. K. E. Hoover. Such carriers, excipients or stabilizers may enhance one or more properties of the active ingredients in the compositions described herein, e.g., bioactivity, stability, bioavailability, and other pharmacokinetics and/or bioactivities.

Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; benzoates, sorbate and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, serine, alanine or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ (polysorbate), PLURONICS™ (nonionic surfactants), or polyethylene glycol (PEG).

In some embodiments, the pharmaceutically acceptable carriers may comprise a non-ionic surfactant having a hydrophilic-lipophilic balance value (HLB value) greater than 5, a solvent, a buffer agent, an oil phase (e.g., medium-chain triglycerides, or soybean oil), or a combination thereof. In some examples, the pharmaceutically acceptable carriers may comprise the non-ionic surfactant, which may form nanoparticles with the stilbene compound. Alternatively, the pharmaceutical composition comprising the stilbene compound may contain no nanoparticles. In one embodiment, the pharmaceutical composition has a pH value in the range of 2.5 to 8.0 when dissolved in the water.

In some embodiments, the pharmaceutically acceptable carriers may further comprise a co-surfactant, a co-solvent, an antioxidant, or any combination thereof. In some instances, the pharmaceutically acceptable carriers may comprise a lipophilic solvent, e.g., triacetin, medium-chain triglycerides, or oil.

In some examples, the pharmaceutical composition provided herein may be free of water. Alternatively or in addition, the pharmaceutical composition provided herein may be free of antioxidants.

### Non-Ionic Surfactants

The pharmaceutical composition disclosed herein may comprise one or more non-ionic surfactants, which may form nanoparticles with the active agent and/or the hydrophilic therapeutic agent as disclosed herein. Non-ionic surfactants used in the present technology are beneficial at least in the formation of nanoparticles in the composition. In some embodiments, nanoparticles have the effect of encapsulating the active agent and/or the hydrophilic therapeutic agent.

Non-ionic surfactants used in any of the pharmaceutical compositions disclosed herein preferably have a hydrophilic-lipophilic balance (HLB) value greater than 5. The non-ionic surfactant is used in the present technology in a ratio as set forth above. Exemplary non-ionic surfactants include, but are not limited to, polysorbate 80 (Tween® 80), polyoxyl 15 hydroxystearate (Solutol® HS-15), polyoxyethylene castor oil derivatives (e.g., polyoxyl 35 castor oil (Kolliphor® ELP), polyoxyl 40 hydrogenated castor oil (Koliphor® RH40) and polyoxyl 60 hydrogenated castor oil (Cremophor® RH60), Polyoxyethylene (12) glyceryl laurate (UNIGLY ML-212), Polyoxyl 20 Stearate (Myrj™ S20), Polyoxyl 40 Stearate (Myrj™ S40), Polyoxyl 12 Cetostearyl Ether (Kolliphor® CS 12) and Polyoxyl 20 Cetostearyl Ether (Kolliphor® CS 20).

In some instances, the non-ionic surfactant may be a polyoxyethylene derivative, also known as Pegylated excipient. In some examples, the polyoxyethylene derivative is a PEG castor oil derivative, which are materials obtained by reacting varying amounts of ethylene oxide with either castor oil or hydrogenated castor oil. Examples include PEG-35 castor oil and PEG-40 hydrogenated Castor Oil. In other examples, the polyoxyethylene derivative is a PEG ester, which can be manufactured by reacting a polyethylene glycol with a fatty acid. Examples include PEG-40 Stearate and PEG-15 Hydroxystearate. In some instances, the ester may be a sorbitan fatty acid ester (e.g., PEG-20 sorbitan monooleate, PEG-40 sorbitan monooleate, PEG-60 sorbitan monooleate, PEG-80 sorbitan monooleate, PEG-20 sorbitan isostearate, PEG-30 sorbitan tetraoleate, PEG-40, -60 sorbitan tetraoleate, PEG-40 sorbitan diisostearate, or PEG-60 sorbitan tetrastearate), an alkyl glyceride (e.g., PEG-8 Caprylic/capric glycerides, PEG-32 hydrogenated palm glycerides, or PEG-32 Lauroyl glycerides), or an alkyl ether (e.g., PEG-6 cetostearyl ether, PEG-12 cetostearyl ether, PEG-20 cetostearyl ether, PEG-10 cetyl ether, PEG-20 cetyl ether, PEG-4 lauryl ether, PEG-23 lauryl ether, PEG-2 oleyl ether, PEG-10 oleyl ether, PEG-20 oleyl ether, PEG-2 stearyl ether, PEG-10 stearyl ether, PEG-21 stearyl ether, or PEG-100 stearyl ether). In yet other instances, the ester may be a laurate (e.g., PEG-2 laurate, PEG-4 laurate, PEG-6 laurate, PEG-8 laurate. PEG-9-14 laurate, PEG-20 laurate, PEG-32-150 laurate, or PEG-12 glyceryl laurate), a dilaurate (e.g., PEG-2 dilaurate, PEG-4 dilaurate, or PEG-6-150 dilaurate), or a stearate (e.g., PEG-2 stearate, PEG-3 stearate, PEG-4 stearate, PEG-4 isostearate, PEG-5-7 stearate, PEG-6-8 isostearate, PEG-8 stearate, PEG-9 stearate, PEG-10 stearate, PEG-10-isostearate, PEG-12 isostearate, PEG-12-18 stearate, PEG-20 stearate, PEG-23-45 stearate, PEG-40 stearate, PEG-50 stearate, PEG-100 stearate, PEG-75-150 stearate, or PEG-6 and PEG-32 palmitostearate), a glyceryl stearate (e.g., Glyceryl stearate/PEG-40 stearate, Glyceryl stearate/PEG-100 stearate, PEG-120 glyceryl stearate, PEG-20 methyl glucose sesquistearate, or PEG-25 propylene glycol stearate), a distearate (e.g., PEG-2 distearate, PEG-3-120 distearate, PEG-150 distearate, or PEG-175 distearate), a hydroxystearate (e.g., PEG-15 hydroxystearates).

In some instances, the non-ionic surfactant may be a castor oil derivative (e.g., PEG-35 castor oil or PEG-40 castor oil) or a hydrogenated castor oil (e.g., PEG-40 hydrogenated castor oil, PEG-54 hydrogenated castor oil, or PEG-60 hydrogenated castor oil).

Additional examples of non-ionic surfactants include PEG-15 cocamine, Vitamin E polyethylene glycol succinate, PEG-75 lanolin, and PEG-120 methyl glucose dioleate.

### Nanoparticles and Preparations Thereof

In some embodiments, the pharmaceutical compositions disclosed herein may comprise nanoparticles formed by one or more non-ionic surfactants as disclosed herein and one or more stilbene compounds as also disclosed herein (e.g., 4-[(E)-2-(3,5-dihydroxyphenyl)ethenyl]benzene-1,3-diol).

In some embodiments, the nanoparticles may comprise the non-ionic surfactant and the stilbene compound at a suitable weight ratio, which lead to suitable particle sizes and/or suitable polydispersity index (PDI) values. For example, a suitable weight ratio between the stilbene compound and the non-ionic surfactant may range from about 1:1-1:1000, for example, between about 1:1-1:800, between about 1:1-1:600, between about 1:1-1:500, between about 1:1-1:200, or between about 1:1-1:100. In other examples, the suitable weight ratio between the stilbene compound and the non-ionic surfactant ranges from 1:5 to 1:1000, for example, between 1:5-1:150, between 1:5-1:200, between 1:5-1:300, between 1:5-1:400, between 1:5-1:500, between about 1:8-1:10, between about 1:8-1:20, between about 1:8-1:40, between about 1:8-1:100, between 1:8-1:150, between 1:8-1:200, between 1:8-1:300, between 1:8-1:400, between 1:8-1:500, between about 1:10-1:20, between about 1:10-1:40, between about 1:10-1:100, between 1:10-1:150, between 1:10-1:200, between 1:10-1:300, between 1:10-1:400, between 1:10-1:500, between about 1:20-1:40, between about 1:20-1:100, between 1:20-1:150, between 1:20-1:200, between 1:20-1:300, between 1:20-1:400, between 1:20-1:500, between about 1:40-1:100, between 1:40-1:150, between 1:40-1:200, between 1:40-1:300, between 1:40-1:400, between 1:40-1:500, between 1:100-1:150, between 1:100-1:200, between 1:100-1:300, between 1:100-1:400, between 1:100-1:500, or between 1:150-1:500.

In some instances, the pharmaceutical compositions disclosed herein may comprise nanoparticles having an average size of up to 200 nm, for example, up to 150 nm, up to 100 nm, up to 50 nm, or up to 20 nm. In some examples, the average size of the nanoparticles may range from 5-200 nm, for example, 5-150 nm, 5-100 nm, 5-50 nm, or 5-20 nm.

Alternatively or in addition, the nanoparticles in the pharmaceutical compositions may have a suitable PDI value, for example, up to 0.4. In some examples, the PDI value may range from 0.05-0.4, e.g., 0.05-0.35, 0.05-0.3, 0.05-0.25, 0.05-0.2, 0.05-0.15, or 0.05-0.1.

Any of the nanoparticles disclosed herein can be prepared by conventional methods or as disclosed herein. One example is provided below. A suitable amount of an active agent or a hydrophilic therapeutic agent may be mixed with a suitable solvent (e.g., alcohols (e.g., methanol, ethanol, propanol), acetonitrile, chlorinated solvents (e.g., dichloromethane, chloroform) diethyl ether, and ethyl acetate) and the mixture can be stirred (e.g., at 150-500 rpm) at a suitable temperature until the active agent or the hydrophilic therapeutic agent as disclosed herein is full dissolved in the solvent to form a solution. A suitable amount of a pharmaceutically acceptable non-ionic surfactant (e.g., those disclosed herein) can be added to the solution. The resultant mixture can be stirred (e.g., at 100-300 rpm) under a suitable temperature to volatilize the solvent. Once the solvent is completely volatilized, a suitable amount of a pharmaceutically acceptable aqueous solution (e.g., normal saline) can be added to the mixture to produce micelles having the active agent or hydrophilic agent encapsulated. The resultant particles can be filtered through a suitable filter (e.g., a 0.2 μm filter) and the filtered solution comprising drug-containing micelles can be stored in the dark in a refrigerator for future use.

Additional details on the preparation of the micelles of the present technology can be found in U.S. Pat. No. 10,610,496, the relevant disclosures of which are incorporated by reference herein for the subject matter and purpose referenced herein.

### Additional Components of Pharmaceutical Compositions

In embodiments, the pharmaceutical composition disclosed herein may further comprise a co-solvent (e.g., to increase the solubility of drugs), a suspending agent (e.g., to reduce the sedimentation rate of drugs), an oil phase excipient (e.g., to increase the stability of the pharmaceutical composition and the solubility of drugs), an antimicrobial preservative, or a combination thereof.

In some instances, the co-solvent can comprise polyethylene glycol, propylene glycol, ethanol, and other co-solvents, or a combination thereof. Alternatively or in addition, the suspending agent may comprise sodium alginate, glycerol, carboxymethyl cellulose sodium, mannitol, and other suspending agents, or a combination thereof.

In some instances, the oil phase excipient may comprise unsaturated fatty acids, glycerol, triglycerides, and other oil phase excipients, or a combination thereof. For example, the oil phase excipient may comprise one or more unsaturated fatty acids, which may be oleic acid, castor oil, sesame oil, cottonseed oil, soybean oil, safflower oil, corn oil, and other unsaturated fatty acids, or a combination thereof. In some examples, the oil phase excipient may comprise triglycerides, which may be medium chain triglycerides.

In some embodiments, the pharmaceutical composition disclosed herein may comprise a local anesthetic. Examples include, but are not limited to, amides, para-aminobenzoic acid esters, and amino ethers, or a combination thereof. In some examples, the local anesthetic comprises amides, which may bedibucaine, lidocaine, mepivacaine HCl, bupivacaine HCl, pyrrocaine HCl, Prilocaine HCl, digammacaine, and oxethazaine, or a combination thereof. In other examples, the local anesthetic comprises para-aminobenzoic acid esters, which may be butacaine, dimethocaine, and tutocaine, or a combination thereof. In yet other examples, the local anesthetic comprises amino ethers, which may be quinisocaine and pramocaine, or a combination thereof.

Alternatively or in addition, the pharmaceutical composition disclosed herein may comprise one or more antioxidant. Examples include, but are not limited to, beta-carotene, lutein, lycopene, bilirubin, vitamin A, vitamin C (ascorbic acid), vitamin E, citric acid, sodium thiosulfate, Propyl gallate, uric acid, nitric oxide, nitroxide, pyruvate, catalase, superoxide dismutase, glutathione peroxidases, N-acetyl cysteine, and naringenin, or a combination thereof. Alternatively, the pharmaceutical composition is free of antioxidants.

Alternatively or in addition, the pharmaceutical compositions provided herein may further comprise a second active agent, which is not a stilbene compound disclosed herein. In some instances, the second active agent may be encapsulated in nanoparticles.

In some embodiments, the second active agent may comprise a hydrophilic therapeutic agent, for example, green tea extract, epicatechin, epicatechin gallate, epigallocatechin, gallocatechin gallate, gallocatechin, catechin gallate, catechin, epigallocatechin gallate (EGCG), caffeine, carnitine, L-carnitine, synephrine, chlorogenic acid, or a combination thereof.

In some embodiments, the pharmaceutical compositions described herein may be formulated as a topical formulation, for example, a cream, lotion, or gel for topical application. Such cream, lotion, or gel may be formulated using ingredients known in the art to be appropriate for topical medications. In some examples, the topical formation may be in the form of a transdermal patch.

In other embodiments, the pharmaceutical compositions described herein may be formulated as an injectable formulation (e.g., for intradermal injection). A sterile injectable composition, e.g., a sterile injectable aqueous or oleaginous suspension, can be formulated according to techniques known in the art using suitable dispersing or wetting agents (such as Polysorbate 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

In some embodiments, the pharmaceutical composition is in a form of non-oral dosage form, and the subject is a human. For example, the composition formulated for injection can be in the form of powder (e.g., lyophilized powder), sterilized suspension, injectable solution, injectable emulsion, or intravenous fluid. In some examples, the composition may be placed in a microneedle device for injection. Alternatively, the composition may be formulated for transdermal administration, e.g., in a form of ointment, lotion, liniment, cream, gel, dressing, emulsion, film, patch, poultice, cataplasm, or topical powder.

### III. Method for Reducing Hyperpigmentation

Any of the stilbene compound (e.g., 5-[(E)-2-(4-hydroxyphenyl)ethenyl]benzene-1,3-diol derivatives such as 4-[(E)-2-(3,5-dihydroxyphenyl)ethenyl]benzene-1,3-diol) or a pharmaceutical composition as disclosed herein can be used to reduce hyperpigmentation in a subject such as a human patient in need of the treatment, for example, to treat, delay the onset, or alleviate hyperpigmentation. Hyperpigmentation refers to any patch of skin that looks darker than natural skin tone of the same subject due to overproduction of the brown pigment melanin, which may be triggered by a specific event or cause, for example, sun exposure, inflammation, or hormone effects. Hyperpigmentation can be seen in age spots or sunspots.

To practice the method disclosed herein, an effective amount of the pharmaceutical composition described herein can be administered to a subject (e.g., a human) in need of the treatment via a suitable route, for example, orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

In some embodiments, the pharmaceutical composition may be administered to a subject such as a human subject by topical administration or intradermal injection. In some instances, the 5-[(E)-2-(4-hydroxyphenyl)ethenyl]benzene-1,3-diol or the pharmaceutical composition comprising such may be applied to a skin site where reduction of hyperpigmentation is needed. As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

For the purpose of the present disclosure, the appropriate dosage of the pharmaceutical composition as described herein will depend on the specific active agents and optionally hydrophilic agents employed, the type and severity of the disease/disorder, whether the composition is administered for preventive or therapeutic purposes, previous therapy, the patient's clinical history and response to the active agents, and the discretion of the attending physician. Typically, the clinician will administer a pharmaceutical composition disclosed herein, until a dosage is reached that achieves the desired result.

In some embodiments, the desired result is the reduction or elimination hyperpigmentation on a skin site, which may be achieved by reducing the level of melanin in skin cells. Methods of determining whether a dosage resulted in the desired result would be evident to one of skill in the art. The particular dosage regimen, i.e., dose, timing and repetition, used in the method described herein will depend on the particular subject and that subject's medical history. Treatment efficacy for a target disease/disorder can be assessed by methods well-known in the art.

In some examples, the stilbene compound such as 4-[(E)-2-(3,5-dihydroxyphenyl)ethenyl]benzene-1,3-diol may be applied to a skin site (e.g., topical such as via a transdermal patch or via intradermal injection) where reduction of hyperpigmentation is needed at a suitable dose, for example, at 0.01-100 mg/cm² based on the treatment area or 0.01-100 mg/kg based on the subject's body weight.

In some instances, the subject such as a human subject may have hyperpigmentation caused by sun exposure. Such a subject may have sun spots, age spots, liver spots, or solar lentigines. An effective amount of the stilbene compound such as a 5-[(E)-2-(4-hydroxyphenyl)ethenyl]benzene-1,3-diol derivative (e.g., 4-[(E)-2-(3,5-dihydroxyphenyl)ethenyl]benzene-1,3-diol) or a pharmaceutical composition comprising such as disclosed herein may be applied to the sun spots, age spots, liver spots, or solar lentigines skin areas of the subject (e.g., a human patient) to reduce hyperpigmentation.

In some instances, the subject such as a human patient for the treatment provided herein may have post-inflammatory hyperpigmentation (PIH), which can be caused by melanocytes producing too much melanin in response to inflammation, injury or certain procedures.

In other instances, the subject such as a human patient may have melasma, a skin condition most commonly occur on the face. In some examples, the subject is a human pregnant woman having melasma.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that delays or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Any of the pharmaceutical compositions disclosed herein may be administered to the subject 1-4 times every day for a suitable period of time, for example, 1 month to 1 year. Duration of the treatment may depend on the treatment results. In some instances, the treatment can be terminated when hyperpigmentation at a skin site is reduced to a certain level so that the involved skin patch has natural skin tone. The treatment may resume when hyperpigmentation occurs again.

### IV. Kits for Use in Reducing Hyperpigmentation

The present disclosure also provides kits for use in treating or alleviating hyperpigmentation in a subject who needs the treatment. Such kits can include one or more containers comprising the pharmaceutical composition disclosed herein. In some instances, the pharmaceutical composition may be co-used with a second therapeutic agent.

In some embodiments, the kit can comprise instructions for use in accordance with any of the methods described herein. The included instructions can comprise a description of administration of the pharmaceutical composition, and optionally the second therapeutic agent, to treat, delay the onset, or alleviate hyperpigmentation in a subject. The kit may further comprise a description of selecting an individual suitable for treatment based on identifying whether that individual has hyperpigmentation, e.g., following a routine procedure.

The instructions relating to the use of the pharmaceutical composition disclosed herein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating hyperpigmentation. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following descriptions are merely certain preferred embodiments of the present disclosure and are not intended to limit the scope of patent application of the present disclosure. Therefore, any alteration or modification that does not depart from the spirits disclosed herein should be included within the scope of patent application of the present disclosure.

### Example 1. Water-Free 4-[(E)-2-(3,5-dihydroxyphenyl)ethenyl]benzene-1,3-diol-ELP Formulations

### Preparation

Take the desired content of 4-[(E)-2-(3,5-dihydroxyphenyl)ethenyl]benzene-1,3-diol and dissolve it in a certain volume of ethyl acetate and stirred at room temperature until 4-[(E)-2-(3,5-dihydroxyphenyl)ethenyl]benzene-1,3-diol dissolved completely. Polyoxyl-35-castor oil (Kolliphor® ELP, or ELP) was then added and stirred to for a water free 4-[(E)-2-(3,5-dihydroxyphenyl)ethenyl]benzene-1,3-diol-ELP formulation.

Table 1 provides the components of three 4-[(E)-2-(3,5-dihydroxyphenyl)ethenyl]benzene-1,3-diol-ELP formulations with different 4-[(E)-2-(3,5-dihydroxyphenyl)ethenyl]benzene-1,3-diol-ELP weight ratios as indicated.

TABLE 1
Water-Free 4-[(E)-2-(3,5-dihydroxyphenyl)ethenyl]benzene-1,3-diol-ELP
Formulations

| | 4-[(E)-2-(3,5- | 4-[(E)-2-(3,5- | |
|---|---|---|---|
| | dihydroxyphenyl)ethenyl]benzene- | dihydroxyphenyl) | |
| | 1,3-diol:ELP | ethenyl]benzene- | ELP |
| Group | (weight ratio) | 1,3-diol (g) | (g) |
| A | 1:1.6 | 2.5 g | 4 g |
| B | 1:1.33 | 3.0 g | 4 g |
| C | 1:1 | 4.0 g | 4 g |

### Appearance Analysis

After placing the 4-[(E)-2-(3,5-dihydroxyphenyl)ethenyl]benzene-1,3-diol-ELP formulations listed in Table 1 on stand for 1 day, the appearances of the formulations were observed with naked eyes. Group A (4-[(E)-2-(3,5-dihydroxyphenyl)ethenyl]benzene-1,3-diol:ELP of 1:1.6) presented a uniform composition without any precipitation; Group B showed a small amount of precipitation; and Group C was very turbid. Group A remained stable, clear, and free of precipitation after a period for up to 5 days.

### Example 2. 4-[(E)-2-(3,5-dihydroxyphenyl)ethenyl]benzene-1,3-diol-ELP Formulations Comprising Water and/or Ascorbic Acid

### Preparation

In this example, all samples were prepared as described in Example 1, except that water and/or 0.1% ascorbic acid were added to form aqueous solutions. Details of the formulations tested in this example are provided in Table 2 below. In all groups, the ELP concentration was approximately 20% (wt %).

TABLE 2
4-[(E)-2-(3,5-dihydroxyphenyl)ethenyl]benzene-1,3-diol-ELP
Formulations with Water and/or Ascorbic Acid

| | 4-[(E)-2-(3,5- | 4-[(E)-2-(3,5- | | |
|---|---|---|---|---|
| | dihydroxyphenyl) | dihydroxyphenyl) | | |
| | ethenyl] | ethenyl] | ELP | Anti- |
| | benzene-1,3-diol:ELP | benzene-1,3-diol | (mg/ | oxidant |
| Group | (weight ratio) | (mg/mL) | mL) | (mg/mL) |
| D | 1:20 | 10 | 20 | 0.1% |
| | | | | ascorbic |
| | | | | acid |
| E | 1:13.3 | 15 | 20 | 0.1% |
| | | | | ascorbic |
| | | | | acid |
| F | 1:10 | 20 | 20 | 0.1% |
| | | | | ascorbic |
| | | | | acid |
| G | 1:8 | 25 | 20 | 0.1% |
| | | | | ascorbic |
| | | | | acid |

### Appearance Analysis

The appearances of the samples listed in Table 2 were observed with naked eyes. The color of the solutions became darker as the concentration of 4-[(E)-2-(3,5-dihydroxyphenyl)ethenyl]benzene-1,3-diol increased. The appearances of the prepared Group D to Group G samples were uniform as clear solutions.

### Stability of Pharmaceutical Compositions by Accelerated Stability Test

The normal storage condition of pharmaceutical compositions is at 2-8° C. An accelerated stability test was performed on the samples listed in Table 2 above. Briefly, the samples listed in Table 2 above were placed in an environment having a relatively high temperature and relatively high humidity (temperature 25° C.±2° C., relative humidity 60%5%) and maintenance of nanoparticles in the samples was examined.

The content of 4-[(E)-2-(3,5-dihydroxyphenyl)ethenyl]benzene-1,3-diol in the nanoparticles was determined by high performance liquid chromatography (HPLC; e.g., HPLC-UV) and defined as the drug content after accelerated stability test. The percentage of the drug content was calculated by dividing the drug content after accelerated stability test by the initial drug content. A percentage of drug content equal to or greater than 95% indicates good stability of the samples being tested.

The accelerated stability test results of samples Group D to Group G were shown in Table 3. Group D, Group E, Group F and Group G were stored at 25° C.±2° C. for 24 hours, one week, and two weeks. After one week, the percentage of 4-[(E)-2-(3,5-dihydroxyphenyl)ethenyl]benzene-1,3-diol drug content in every sample was greater than 95% and did not show a significant trend of decrease comparing to the initial drug content. This result indicates that the pharmaceutical compositions have excellent stability.

TABLE 3
Stability of 4-[(E)-2-(3,5-dihydroxyphenyl)ethenyl]benzene-1,3-diol
Formulations by Accelerated Stability Test

| | Theoretical | | Time point | | |
|---|---|---|---|---|---|
| | Drug | | (Actual Configuration | | |
| | Concen- | | | | |
| | tration | | Concentration) | | |
| Group | (mg/mL) | Drug Content | T0 | 7 days | 14 days |
| D | 10 mg/mL | 4-[(E)-2-(3,5- | 10.462 | 9.588 | 9.479 |
| | | dihydroxyphenyl)ethenyl | | | |
| | | ]benzene-1,3-diol Conc. | | | |
| | | (mg/mL) | | | |
| | | 4-[(E)-2-(3,5- | 104.62 | 95.88 | 94.79 |
| | | dihydroxyphenyl)ethenyl | | | |
| | | ]benzene-1,3-diol (%) | | | |
| E | 15 mg/mL | 4-[(E)-2-(3,5- | 15.660 | 14.524 | 14.755 |
| | | dihydroxyphenyl)ethenyl | | | |
| | | ]benzene-1,3-diol Conc. | | | |
| | | (mg/mL) | | | |
| | | 4-[(E)-2-(3,5- | 104.40 | 96.83 | 98.37 |
| | | dihydroxyphenyl)ethenyl | | | |
| | | ]benzene-1,3-diol (%) | | | |
| F | 20 mg/mL | 4-[(E)-2-(3,5- | 20.001 | 19.649 | 19.864 |
| | | dihydroxyphenyl)ethenyl | | | |
| | | ]benzene-1,3-diol Conc. | | | |
| | | (mg/mL) | | | |
| | | 4-[(E)-2-(3,5- | 100.01 | 98.25 | 99.32 |
| | | dihydroxyphenyl)ethenyl | | | |
| | | ]benzene-1,3-diol (%) | | | |
| G | 25 mg/mL | 4-[(E)-2-(3,5- | 24.469 | 23.776 | 24.480 |
| | | dihydroxyphenyl)ethenyl | | | |
| | | ]benzene-1,3-diol Conc. | | | |
| | | (mg/mL) | | | |
| | | 4-[(E)-2-(3,5- | 97.88 | 95.10 | 97.92 |
| | | dihydroxyphenyl)ethenyl | | | |
| | | ]benzene-1,3-diol (%) | | | |

Impact of antioxidant on the stability of 4-[(E)-2-(3,5-dihydroxyphenyl)ethenyl]benzene-1,3-diol formulations was investigated. The experimental design is listed in Table 4.

TABLE 4
4-[(E)-2-(3,5-dihydroxyphenyl)ethenyl]benzene-1,3-diol Formulations with or
without Antioxidant

| | 4-[(E)-2-(3,5- | 4-[(E)-2-(3,5- | | |
|---|---|---|---|---|
| | dihydroxyphenyl)ethenyl] | dihydroxyphenyl)ethenyl] | | |
| | benzene-1,3-diol: ELP | benzene-1,3-diol | ELP | Antioxidant |
| Group | (weight ratio) | (mg/mL) | (mg/mL) | (mg/mL) |
| H | 1:8 | 25 mg/mL | 20% | 0.1% ascorbic |
| | | | | acid |
| I | 1:8 | 25 mg/mL | 20% | — |

The accelerated stability test results of Group H and Group I are shown in Table 5 and Table 6, respectively.

TABLE 5
Accelerated Stability Test of Group H Formulation

| | | T0 | | 1M | |
|---|---|---|---|---|---|
| Group | Test Item. | L | A | L | A |
| H | Assay | 99.92 | | 98.22 | 98.09 |
| | 4-[(E)-2-(3,5- | | | | |
| | dihydroxyphenyl)ethenyl]benzene- | | | | |
| | 1,3-diol | | | | |

Storage Conditions: 5° C. ± 3° C. (Long-term or “L”); 25° C. ± 2° C./60% RH ± 5% RH(Accelerated or “A))

TABLE 6
Accelerated Stability Test of Group I Formulation

| | | T0 | | 1M | |
|---|---|---|---|---|---|
| Group | Test Item. | L | A | L | A |
| I | Assay | 99.38 | | 96.59 | 98.25 |
| | 4-[(E)-2-(3,5- | | | | |
| | dihydroxyphenyl)ethenyl]benzene- | | | | |
| | 1,3-diol | | | | |

Storage Conditions: 5° C. ± 3° C. (Long-term or “L”); 25° C. ± 2° C./60% RH ± 5% RH (Accelerated or “A”)

### Analysis of Particle Size

A particle size analyzer was used to determine the distribution of particle diameters. If the particle diameter of the pharmaceutical composition, after being analyzed by a particle analyzer, is smaller than 250 nm and the PDI value is less than 0.4, it corresponds to the solution of the pharmaceutical composition being seen as clear and transparent when observed by the naked eye, and the light beam able to be observed when the solution of the pharmaceutical composition is shined by a laser. The results showed that Group H and Group I were formulated to have similar particle sizes (Table 7 and Table 8).

TABLE 7
Particle Size Analysis of Group H Formulation

| Group H | Size | | Average | |
|---|---|---|---|---|
| Sample Name | (nm) | PDI | Size (nm) | PDI |
| 2x dilute | 25.48 | 0.1266 | 26.27 | 0.2216 |
| | 26.54 | 0.2598 | | |
| | 26.79 | 0.2784 | | |
| 5x dilute | 25.26 | 0.1543 | 25.55 | 0.1691 |
| | 25.56 | 0.1708 | | |
| | 25.82 | 0.1823 | | |
| 10x dilute | 20.20 | 0.07485 | 20.25 | 0.0748 |
| | 20.12 | 0.1053 | | |
| | 20.42 | 0.04421 | | |

TABLE 8
Particle Size Analysis of Group I Formulation

| Group I | | | Average | |
|---|---|---|---|---|
| Sample | Size | | Size | |
| Name | (nm) | PDI | (nm) | PDI |
| 2x dilute | 26.31 | 0.276 | 26.84 | 0.2912 |
| | 27.18 | 0.2742 | | |
| | 27.04 | 0.3233 | | |
| 5x dilute | 25.54 | 0.1799 | 30.35 | 0.3437 |
| | 29.03 | 0.3122 | | |
| | 36.49 | 0.5389 | | |
| 10x dilute | 20.16 | 0.1669 | | 0.1254 |
| | 20.34 | 0.1178 | 20.29 | |
| | 20.38 | 0.09158 | | |

The results reported herein show that the tested 4-[(E)-2-(3,5-dihydroxyphenyl)ethenyl]benzene-1,3-diol formulations are stable, regardless of whether antioxidants are added or not.

### Example 3. 4-[(E)-2-(3,5-dihydroxyphenyl)ethenyl]benzene-1,3-diol Inhibit Melanin Production In Vitro

### In Vitro Efficacy Test

B16-F10 cells are a melanoma cell line derived from C57BL/6 mice, which produce melanin and display metastatic behaviors. Thus, B16-F10 cells are widely used to study melanogenesis and depigmentation.

Alpha-melanocyte-stimulating hormone (α-MSH) is a peptide hormone capable of inducing melanin production. In this study, the effects of 4-[(E)-2-(3,5-dihydroxyphenyl)ethenyl]benzene-1,3-diol samples (having different concentrations of 4-[(E)-2-(3,5-dihydroxyphenyl)ethenyl]benzene-1,3-diol) on inhibition of melanin production were investigated. The experimental design is listed in Table 9.

TABLE 9
Experimental Design of the In Vitro Efficacy Test

| | | | Concentration of 4-[(E)-2- | |
|---|---|---|---|---|
| | | | (3,5- | |
| | | α-MSH | dihydroxyphenyl)ethenyl] | |
| Group | Solvent | (0.1 μM) | benzene-1,3-diol | Treatment Time |
| Vehicle | 0.2% | − | − | α-MSH for 24 hours followed by |
| Control | DMSO | + | − | 4-[(E)-2-(3,5- |
| IM01- | | + | 8 ppm | dihydroxyphenyl)ethenyl]benzene- |
| C1 | | | | 1,3-diol samples for 24 hours |
| IM01- | | + | 11.3 ppm | |
| C2 | | | | |
| IM01- | | + | 16 ppm | |
| C3 | | | | |
| IM01- | | + | 22.6 ppm | |
| C4 | | | | |
| IM01- | | + | 32 ppm | |
| C5 | | | | |
| IM01- | | + | 64 pm | |
| C6 | | | | |

### Methods and Materials

### A. Test Conditions

B16-F10 cells were seeded in a 96-well plate at a density of 1×10⁵ cells/well and cultured for 24 hours. The cells were then incubated with 0.1 μM α-MSH for 24 hours to include melanin production. The 4-[(E)-2-(3,5-dihydroxyphenyl)ethenyl]benzene-1,3-diol samples with different concentrations were then added to the cultured cells, which were cultured for an additional 24 hours. No α-MSH was used for the vehicle control group.

### B. Determination of Melanin Content in Each Group

After the cells were washed once with 1 mL of 1×PBS, add 300 μL of 1×Trypsin-EDTA, they were placed in a 37° C. incubator for 5 minutes. Finally, 1 mL of DMEM was added to the cells, and the number of cells were counted by Trypan blue staining.

Cell suspension was centrifuged at 1500 g for 5 minutes at room temperature, followed by supernatant removal and air-dry of the pellet for 15 minutes. 1 N NaOH (containing 10% DMSO) was added to break up the pellet, followed by shaking by ultrasonic waves for 30 minutes and heating in a water bath at 80° C. for 1.5 hours. After cooling down, the suspension was taken and measured for absorbance at 405 nm. Results were calculated by deducting the reagent blank from the absorbance value at 405 nm and divided by the average absorbance value of the α-MSH Group.

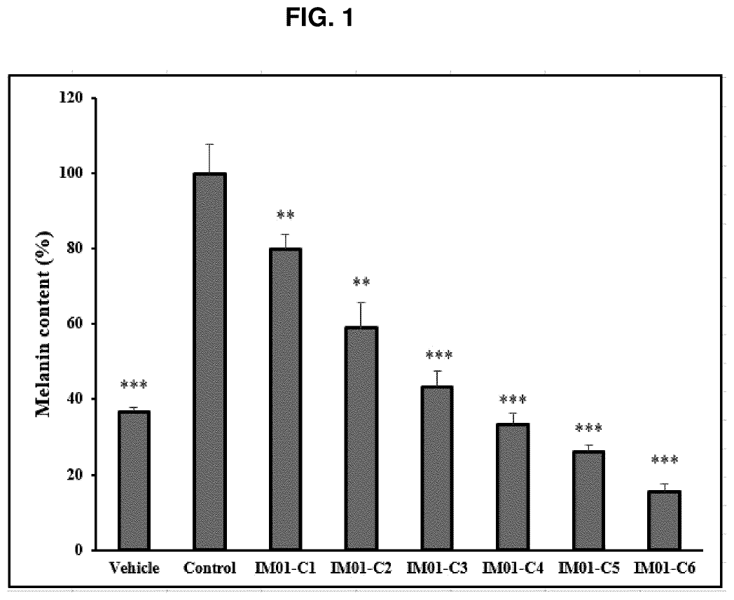

The image and quantification results obtained after extracting the melanin content of each group are shown in FIG. 1 and Table 10 (as used herein, “TA” refers to test article). The EC₅₀ of the 4-[(E)-2-(3,5-dihydroxyphenyl)ethenyl]benzene-1,3-diol formulation was found to be around 15.2 ppm (equal to 0.062 μM). The results showed that the 4-[(E)-2-(3,5-dihydroxyphenyl)ethenyl]benzene-1,3-diol samples tested herein significantly reduced melanin content in a dose-dependent manner as compared to the control group (with α-MSH only). IM01-C4, IM01-C5, and IM01-C6 groups reduced melanin production to levels lower than that of the vehicle group (without α-MSH), suggesting that the 4-[(E)-2-(3,5-dihydroxyphenyl)ethenyl]benzene-1,3-diol samples not only suppressed melanin production induced by α-MSH but also reduced baseline melanin production.

TABLE 10
Melanin Content of Cells Treated by 4-[(E)-2-(3,5-dihydroxyphenyl)
ethenyl]benzene-1,3-diol Samples in In Vitro Efficacy Test
Melanin content (%)

| Group | Vehicle | Control | IM01-C1 | IM01-C2 | IM01-C3 | IM01-C4 | IM01-C5 | IM01-C6 |
|---|---|---|---|---|---|---|---|---|
| TA conc. | 0.2% | 0.2% | 8 | 11.3 | 16 | 22.6 | 32 | 64 |
| (ppm) | DMSO | DMSO | | | | | | |
| α-MSH | − | + | + | + | + | + | + | + |
| Mean | 36.7 | 100 | 79.9 | 58.9 | 43.2 | 33.3 | 26.0 | 15.4 |
| SD | ±1.20 | ±7.71 | ±3.93 | ±6.90 | ±4.37 | ±3.14 | ±2.03 | ±2.32 |

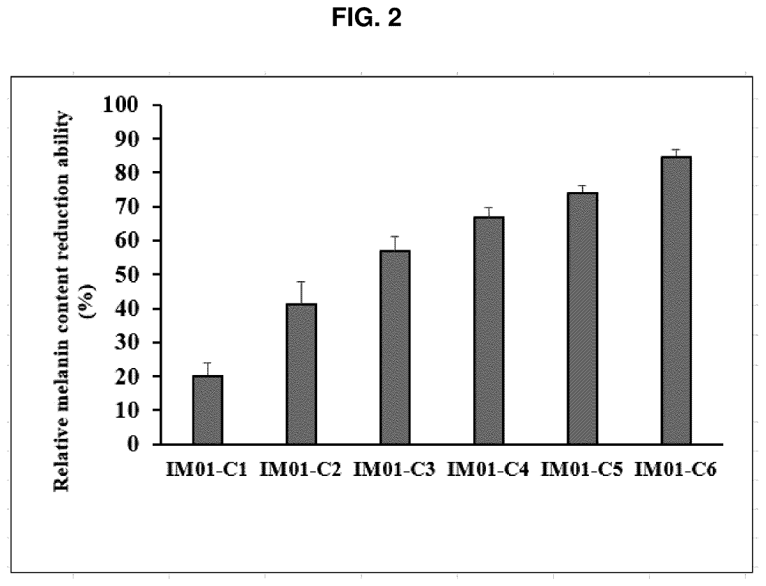

Within the tested concentrations, it was shown that the 4-[(E)-2-(3,5-dihydroxyphenyl)ethenyl]benzene-1,3-diol samples reduce melanin levels in a dose-dependent manner. FIG. 2 and Table 11).

TABLE 11
Melanin Inhibition Ability of the 4-[(E)-2-(3,5-dihydroxyphenyl)ethenyl] benzene-1,3-diol
Formulations
Melanin Content Inhibition Ability (%)

| Group | IM01-C1 | IM01-C2 | IM01-C3 | IM01-C4 | IM01-C5 | IM01-C6 |
|---|---|---|---|---|---|---|
| TA conc. | 8 | 11.3 | 16 | 22.6 | 32 | 64 |
| (ppm) | | | | | | |
| Mean | 20.1 | 41.1 | 56.8 | 66.7 | 74 | 84.6 |
| SD | ±3.93 | ±6.90 | ±4.37 | ±3.14 | ±2.03 | ±2.32 |

### Example 4. 4-[(E)-2-(3,5-dihydroxyphenyl)ethenyl]benzene-1,3-diol Reduces Melanin Levels In Vivo

This example investigates the therapeutic effect of 4-[(E)-2-(3,5-dihydroxyphenyl)ethenyl]benzene-1,3-diol compositions for reducing melanin in vivo via different administration routes or different dosing schedules.

(A) In Vivo Efficacy of 4-[(E)-2-(3,5-dihydroxyphenyl)ethenyl]benzene-1,3-diol Compositions Via Different Administration Routes

Five-week-old male HRM-2 mice, which were melanin-possessing hairless mice, were used in the study. The experiment tested a total of 20 mice using conventional diets in the adaptation period, the induction period, and the test period. After 7 days of adaptation, the mice were randomly divided into a control group and a UVB-c group according to body weight and body weight changes, so that no statistical difference in average body weight and body weight changes were noticeable among the groups. The UVB-c group was subjected to 150 mJ/cm²/day of UVB irradiation with a frequency of irradiating the back of the mice three times a week. After four weeks (the 29^{th} day of UV irradiation, the first day of the test period) of irradiation, the back of the mice became significantly black, which were then divided into 4 groups according to the average body weight and body weight changes in the UVB-induced group: UVB-c group (UVB control), IM01-ID group (intradermal injection), IM01-Topical group (topical application), and color cream group (“three-in-one” commercial cream, containing 5% hydroquinone, 0.1% dexamethasone, and 0.1% tretinoin. See below). The experimental design is shown in Table 12.

TABLE 12
Experimental Design of the In Vivo Efficacy Test

| | UVB | | | Total | |
|---|---|---|---|---|---|
| | Irradiation | Concentration | | Dose | |
| | Frequency & | of Test | Dose | Level/ | Dosing |
| Group | Dose | Article | Level | Per Mice | Frequency |
| Control | — | — | — | — | — |
| UVB-c | 150 mJ/cm², | — | — | — | — |
| (UVB-Control) | every 2 days for | | | | |
| IM01-ID | 7 weeks | 20 mg/mL | 0.5 | 3 mg | Once a |
| (Intradermal) | | | mg/cm² | | week |
| IM01-Topical | | 20 mg/mL | 0.4-0.5 | 2.4-3 mg | Once a day |
| | | | mg/cm² | | |
| Color cream | | — | 0.7-1.0 | 4.2-6.0 mg | Once a day |
| (“Three-in-one” | | | mg/cm² | | |
| commercial | | | | | |
| cream) | | | | | |

As shown in Table 12, the IM01—ID group was administered by intradermal injection. Injection frequency was once a week, three times in total, with an average injection area of 6 cm² skin each time. The IM01—Topical group and the color cream group were administered by smearing once a day for a total of 21 days. The color cream group was a commercially available three-in-one whitening cream (with 5% hydroquinone, 0.3% tretinoin and 0.3% dexamethasone), which was used here to compare the efficacy with the formulation of the present disclosure. During the treatment period, the backs of the mice were irradiated with 150 mJ/cm²/day of UVB three times a week until sacrificed. It should be noted that the timing of UVB exposure and drug administration were staggered.

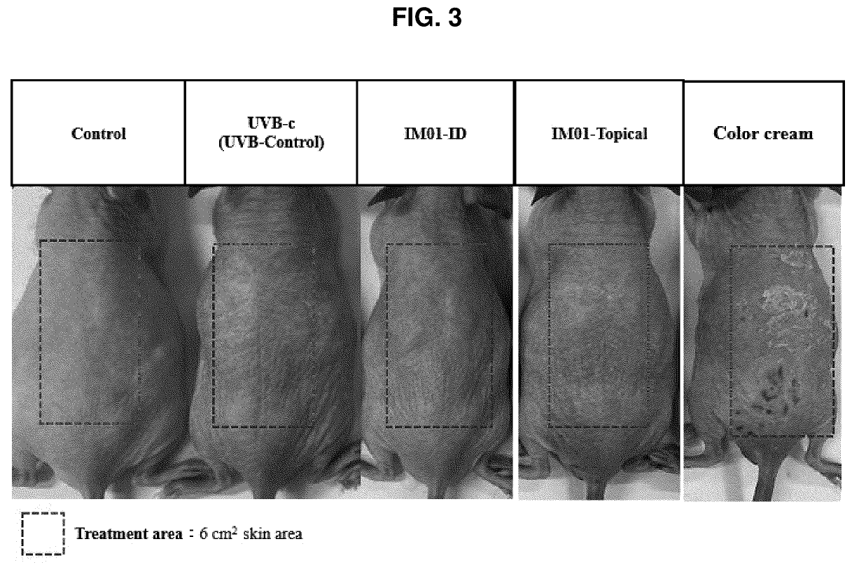

In this experiment, UVB was used to irradiate HRM-2 mice to produce melanin precipitation and aging, so as to evaluate the whitening efficacy of IM01 and compare it with the commercially available three-in-one whitening cream. As shown in FIG. 3, the skin color of the UVB-c group was darker than that of the control group, indicating melanin deposition.

The skin of the UVB-c mice was dry and wrinkled, and the cutin was thickened. After treatment by the 4-[(E)-2-(3,5-dihydroxyphenyl)ethenyl]benzene-1,3-diol compositions as indicated, by intradermal injection or smearing, the skin color of the mice in the IM01—ID group and the IM01—Topical group became whiter and brighter than that of the UVB-c group, and the amount of melanin deposition in both groups was lower than that of the UVB-c group. Although the color cream group showed less melanin than the UVB-c group, skin dryness and peeling were observed in all the treated areas. IN some severe cases, wounds, redness, and even darkening of the skin occurred (consistent with the side effects listed in the package insert).

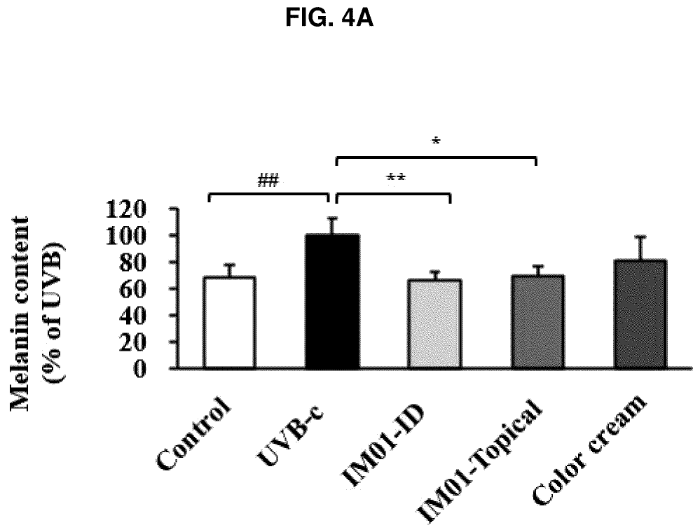

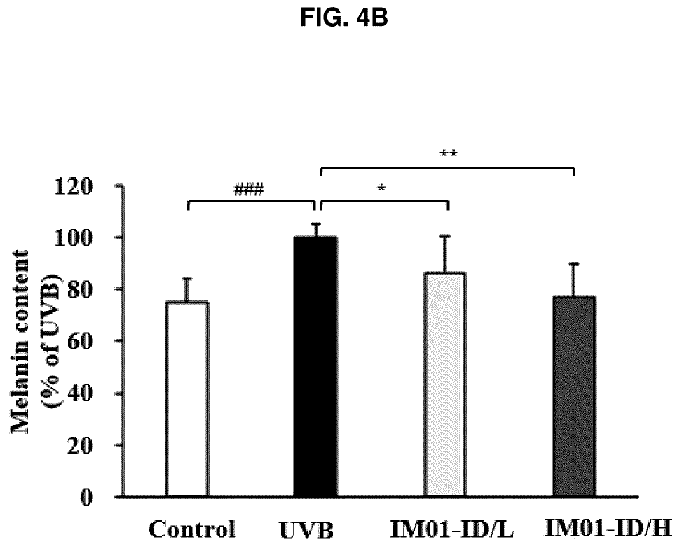

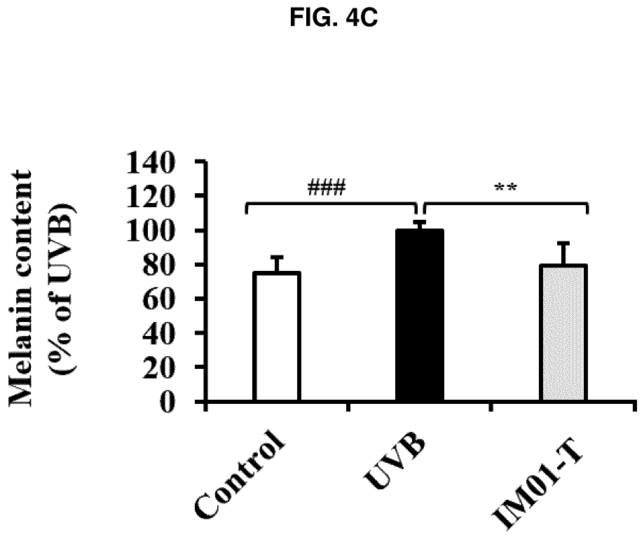

After sacrificing the mice on day 22, the skin of treatment area was collected and subjected to heat extraction with 0.4 mL NaOH for 1 hour. As shown in FIG. 4A and Table 13, the melanin content of the UVB-c group was significantly higher than that of the control group, i.e., by 31.4% (p<0.01), and the IM01—ID group and the IM01—Topical group showed significantly lower melanin levels than the UVB-c group, i.e., by 33.6% (p<0.01) and 30.6% (p<0.05), respectively. The experimental data were expressed as mean±standard deviation (n=3-5), using Student's t-test analysis, #p<0.05, ##p<0.01 compared with control group; *p<0.05, **p<0.01 compared with UVB-c group. In contrast, the skin melanin content of the color cream group appeared lower than that of the UVB-c group but with no statistically significant difference.

TABLE 13
Melanin Inhibition Results of the Formulations in In Vivo Efficacy Test

| | | | | IM01- | Color |
|---|---|---|---|---|---|
| Group | Control | UVB-c | IM01-ID | Topical | cream |
| Melanin Content | 68.6 ± 8.9 | 100.0 ± 12.8 | 66.4 ± 6.0 | 69.4 ± 7.6 | 81.3 ± 17.4 |
| Mean ± SD | | | | | |
| (% of UVB-c) | | | | | |
| Percentage | — | 0.0 ± 12.8 | −33.6 ± 6.0 | −30.6 ± 7.6 | −18.7 ± 17.4 |
| change of melanin | | | | | |
| Mean ± SD | | | | | |
| (% of UVB-c) | | | | | |
| t-test | ## | — | ** | * | — |

#p < 0.05,
##p < 0.01 compared with Control;
*p < 0.05,
**p < 0.01 compared with UVB-c.

In summary, the results from this example demonstrated that 4-[(E)-2-(3,5-dihydroxyphenyl)ethenyl]benzene-1,3-diol compositions, either administered intradermally or topically, significantly reduced UVB-induced hyperpigmentation in HRM-2 mice.

B. In Vivo Efficacy of 4-[(E)-2-(3,5-dihydroxyphenyl)ethenyl]benzene-1,3-diol Composition by Intradermal Injection

The IM01 formulation was investigated for in vivo efficacy via intradermal injection, either twice a week (IM01-ID/L) or once a day (IM01-ID/H). The study design is provided in Table 14 below.

TABLE 14
Experimental Design of In Vivo Efficacy Test via Intradermal Injection
Study Design

| | UVB-irradiated | TA Conc. | Dosing | Dose |
|---|---|---|---|---|
| Study Group | Period | (mg/mL) | route | Frequency |
| Control | — | — | — | — |
| UVB | Pre-induction for | — | — | — |
| IM01-ID/L | 4 weeks + | 23 | ID | Twice a week |
| IM01-ID/H | Treatment period | 23 | ID | Once a day |
| | for 3 weeks | | | |

The results from this study show that IM01 via intradermal injection led to significant whitening as indicated by the reduction of melanin levels at the injection sites in a dose-dependent manner. IM01-ID/L and IM01-ID/H significantly reduced melanin production by 13.9% and 23.1%, respectively. See Table 15 and FIG. 4B.

TABLE 15
Melanin Inhibition Results via Intradermal Injection

| Group | Control | UVB | IM01-ID/L | IM01-ID/H |
|---|---|---|---|---|
| Mean ± SD | 75.0 ± 9.3 | 100.0 ± 4.8 | 86.1 ± 14.5 | 76.9 ± 12.7 |

C. In Vivo Efficacy of 4-[(E)-2-(3,5-dihydroxyphenyl)ethenyl]benzene-1,3-diol Composition by Topical Administration

The IM01 formulation was investigated for in vivo efficacy via topical administration (IM01-T), following the study design provided in Table 16 below.

TABLE 16
Experimental Design of In Vivo Efficacy Test via Topical Administration
Study Design

| | UVB- | | | |
|---|---|---|---|---|
| | irradiated | TA Conc. | Dosing | Dose |
| Study Group | Period | (mg/mL) | route | Frequency |
| Control | — | — | — | — |
| UVB | Pre-induction | — | — | — |
| IM01-T | for 4 weeks + | 45 | Topical | Once a day |
| | Treatment | | | |
| | period for 3 | | | |
| | weeks | | | |

The results from this study show that IM01 via topical administration led to significant whitening as indicated by the reduction of melanin levels at the administration sites. IM01-T significantly reduced melanin production by 21.4%. See Table 17 and FIG. 4C.

TABLE 17
Melanin Inhibition Results via Topical Administration

| Group | Control | UVB | IM01-T |
|---|---|---|---|
| Mean ± SD | 75.0 ± 9.3 | 100.0 ± 4.8 | 79.6 ± 12.5 |

### Example 5. 4-[(E)-2-(3,5-dihydroxyphenyl)ethenyl]benzene-1,3-diol Exhibited Better in Vitro Efficacy as Compared with Other Skin Whitening Compounds

### In Vitro Efficacy Comparison

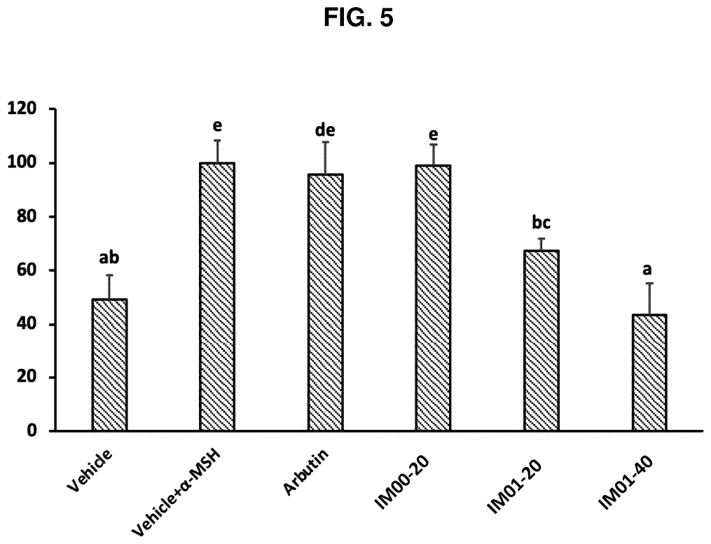

B16-F10 cells were seeded in a 96-well plate. Except the vehicle group, cells of the other groups were cultured in the presence of α-MSH for 0.5 hours first. The compositions for examination were then added to the culture medium and the cells were further cultured for 30 hours. The rest of the experimental conditions and procedures were similar to those described in Example 3. Afterwards, the cells were collected to evaluate the effect of 4-[(E)-2-(3,5-dihydroxyphenyl)ethenyl]benzene-1,3-diol compositions (IM01 Group) and Arbutin and 5-[(E)-2-(4-hydroxyphenyl)ethenyl]benzene-1,3-diol (IM00 Group) compositions on reducing melanin levels. The experimental design is summarized in Table 18 and results shown in both Table 18 and FIG. 5.

TABLE 18
Experimental Design and Results of the In Vitro Efficacy Comparison
Melanin content (%)

| | | Vehicle + | | | | |
|---|---|---|---|---|---|---|
| Group | Vehicle | α-MSH | Arbutin | IM00-20 | IM01-20 | IM01-40 |
| TA conc. | 0.2% | 0.2% | 918.27 | 87.62 | 89.19 | 178.38 |
| (μM) | DMSO | DMSO | | | | |
| α-MSH | − | + | + | + | + | + |
| Mean | 49.1 | 100.0 | 95.8 | 98.9 | 67.4 | 43.5 |
| SD | 9.3 | 8.3 | 12.0 | 7.9 | 4.4 | 11.8 |
| t-test | 0.005 | 1.000 | 0.696 | 0.901 | 0.013 | 0.005 |
| ANOVA | ab | e | de | e | bc | a |

The results show that 4-[(E)-2-(3,5-dihydroxyphenyl)ethenyl]benzene-1,3-diol exhibited better in vitro effects in reducing melanin relative to 5-[(E)-2-(4-hydroxyphenyl)ethenyl]benzene-1,3-diol and arbutin.

### Example 6. In Vitro Efficacy of 4-[(E)-2-(3,5-dihydroxyphenyl)ethenyl]benzene-1,3-diol and Known Skin Whitening Agents

This example aims to evaluate the in vitro effect of 4-[(E)-2-(3,5-dihydroxyphenyl)ethenyl]benzene-1,3-diol compositions (dissolved in 0.2% DMSO at different concentrations as indicated) as relative to known whitening agents arbutin and hydroquinone on melanin production in of B16-F10 cells.

In this experiment, B16-F10 cells were seeded in a 96-well plate and divided into several treatment groups as listed in Table 19 and Table 20 below. Except the vehicle group, cells of the other groups were cultured with α-MSH for 24 hours first. The compositions for investigation (see Tables 19 and 20) were then added to the culture medium and the cells were further cultured for 24 hours. The rest of the experimental operation process was similar to that described in Example 3. Afterwards, the cells were collected to evaluate the effect of 4-[(E)-2-(3,5-dihydroxyphenyl)ethenyl]benzene-1,3-diol (IM01 group), arbutin, and hydroquinone on suppressing melanin.

TABLE 19
Exemplary Experimental Design Comparing
Effects of 4-[(E)-2-(3,5-dihydroxyphenyl)ethenyl]benzene-
1,3-diol with Arbutin and Hydroquinone

| | | | α-MSH | TA Conc. | Treatment |
|---|---|---|---|---|---|
| No. | Group | Vehicle | (0.1 μM) | (μM) | Time |
| 1 | Vehicle | 0.2% | − | − | α-MSH |
| 2 | α-MSH | DMSO | + | − | treat |
| 3 | IM01-60 | | + | 60 | 24 hr + |
| 4 | IM01-120 | | + | 120 | TA treat |
| 5 | Arbutin-450 | | + | 450 | 24 hr |
| 6 | Arbutin-900 | | + | 900 | |
| 7 | Hydroquinone- | | + | 120 | |
| | 120 | | | | |

TABLE 20
Exemplary Experimental Design Comparing Effects of 4-[(E)-2-
(3,5-dihydroxyphenyl)ethenyl]benzene-1,3-diol with Arbutin

| | | | α-MSH | TA Conc. | |
|---|---|---|---|---|---|
| No. | Group | Vehicle | (0.1 μM) | (μM) | Treatment Time |
| 1 | Vehicle | 0.2% | − | − | α-MSH treat |
| 2 | α-MSH | DMSO | + | − | 24 hr + TA |
| 3 | Arbutin-900 | | + | 900 | treat 24 hr |
| 4 | Arbutin-1800 | | + | 1800 | |
| 5 | IM01-30 | | + | 30 | |
| 6 | IM01-60 | | + | 60 | |
| 7 | IM01-120 | | + | 120 | |
| 8 | IM01-180 | | + | 180 | |

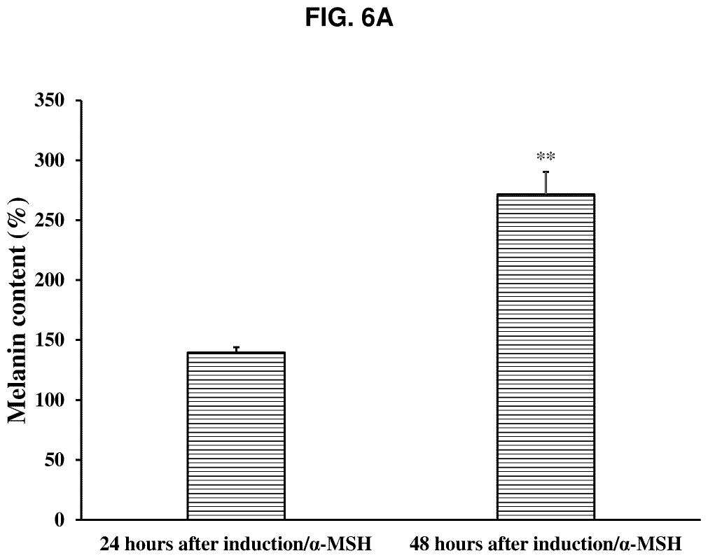

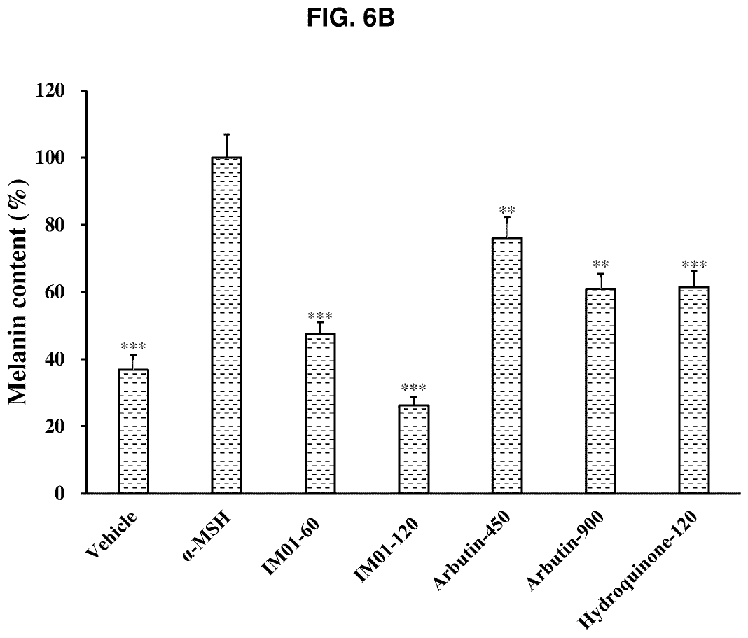

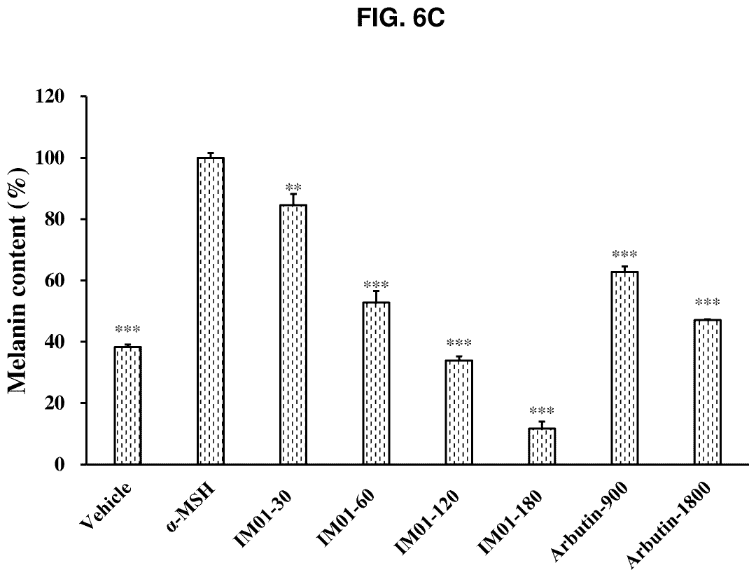

After 24 hours of induction by α-MSH in melanocytes, melanin has increased significantly. FIG. 6A shows that, after 24 hours and 48 hours of induction by α-MSH, the melanin content increase significantly in treated cells as compared with the vehicle control group.

FIGS. 6B-6C and Tables 21-22 show melanin contents of the various treatment groups after culturing with the compositions for investigation for 24 hours. The 4-[(E)-2-(3,5-dihydroxyphenyl)ethenyl]benzene-1,3-diol compositions (IM01 group) significantly reduced melanin contents, for example, at both 60 μM and 120 μM, in a dose-dependent manner. The melanin content of the cells treated by 4-[(E)-2-(3,5-dihydroxyphenyl)ethenyl]benzene-1,3-diol at 120 μM was even lower than that of the vehicle group (with no α-MSH induction), suggesting that 4-[(E)-2-(3,5-dihydroxyphenyl)ethenyl]benzene-1,3-diol can reduce baseline melanin levels (in the absence of α-MSH induction). In addition, the test results also showed that the effect of 4-[(E)-2-(3,5-dihydroxyphenyl)ethenyl]benzene-1,3-diol in reducing melanin (e.g., at 120 μM) was better than that of the whitening agent Arbutin (e.g., at either 450 μM or 900 μM), and the whitening agent hydroquinone (e.g., at 120 μM).

TABLE 21
Inhibition of 4-[(E)-2-(3,5-dihydroxyphenyl)ethenyl]benzene-1,3-diol (IM01
group) on Melanin Contents As Compared with Arbutin and Hydroquinone
Melanin content (%)

| | | | | | | | Hydroquinone- |
|---|---|---|---|---|---|---|---|
| Group | Vehicle | α-MSH | IM01-60 | IM01-120 | Arbutin-450 | Arbutin-900 | 120 |
| TA conc. | − | − | 60 | 120 | 450 | 900 | 120 |
| (μM) | | | | | | | |
| α-MSH | − | + | + | + | + | + | + |
| Mean ± SD | 36.8 ± 4.4 | 100.0 ± 6.9 | 47.6 ± 3.4 | 26.2 ± 2.4 | 76.1 ± 6.3 | 60.9 ± 4.5 | 61.5 ± 4.6 |

TABLE 22
Inhibition of 4-[(E)-2-(3,5-dihydroxyphenyl)ethenyl]benzene-1,3-diol (IM01
group) on Melanin Contents As Compared with Arbutin
Melanin content (%)

| | | | | | | | Arbutin- | Arbutin- |
|---|---|---|---|---|---|---|---|---|
| Group | Vehicle | α-MSH | IM01-30 | IM01-60 | IM01-120 | IM01-180 | 900 | 1800 |
| TA conc. | − | − | 30 | 60 | 120 | 180 | 900 | 1800 |
| (μM) | | | | | | | | |
| α-MSH | − | + | + | + | + | + | + | + |
| Mean ± SD | 38.3 ± 0.8 | 100.0 ± 1.6 | 84.6 ± 3.6 | 52.8 ± 3.8 | 33.9 ± 1.4 | 11.7 ± 2.3 | 62.8 ± 1.8 | 47.1 ± 0.3 |

### Example 7. In Vitro Whitening Efficacy and Toxicity Studies

This example explores the in vitro whitening efficacy and toxicity of 4-[(E)-2-(3,5-dihydroxyphenyl)ethenyl]benzene-1,3-diol in comparison with compounds Arbutin and hydroquinone.

A. In Vitro Efficacy and Toxicity of 4-[(E)-2-(3,5-dihydroxyphenyl)ethenyl]benzene-1,3-diol

Table 23 below lists the study design for evaluating EC50 values of IM01, which contains 4-[(E)-2-(3,5-dihydroxyphenyl)ethenyl]benzene-1,3-diol.

TABLE 23
Study Design of In Vitro EC₅₀ Value Evaluation
Study Design

| Study | | | TA Conc. | | | |
|---|---|---|---|---|---|---|
| Group | Vehicle | α-MSH | (μM) | Treatment Time | Test System | Assay |
| Vehicle | 0.2% | − | − | α-MSH treat 24 hr | B16-F10 cell | 1. Relative |
| α-MSH | DMSO | + | − | + | line | cell viability |
| IM01/4 | | + | 4 | TA treat 24 hr | | 2. Melanin |
| IM01/8 | | + | 8 | | | content |
| IM01/16 | | + | 16 | | | |
| IM01/32 | | + | 32 | | | |
| IM01/64 | | + | 64 | | | |
| IM01/128 | | + | 128 | | | |
| IM01/181 | | + | 181 | | | |
| IM01/256 | | + | 256 | | | |

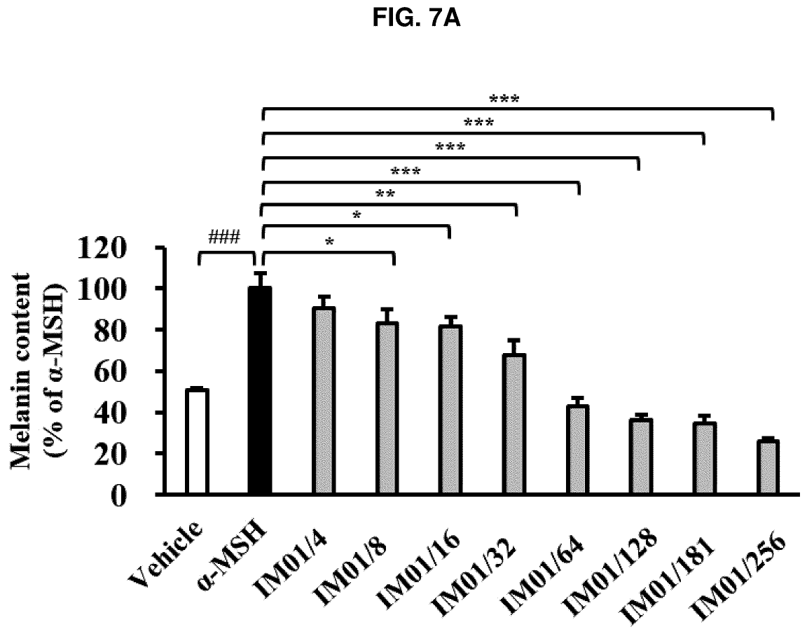

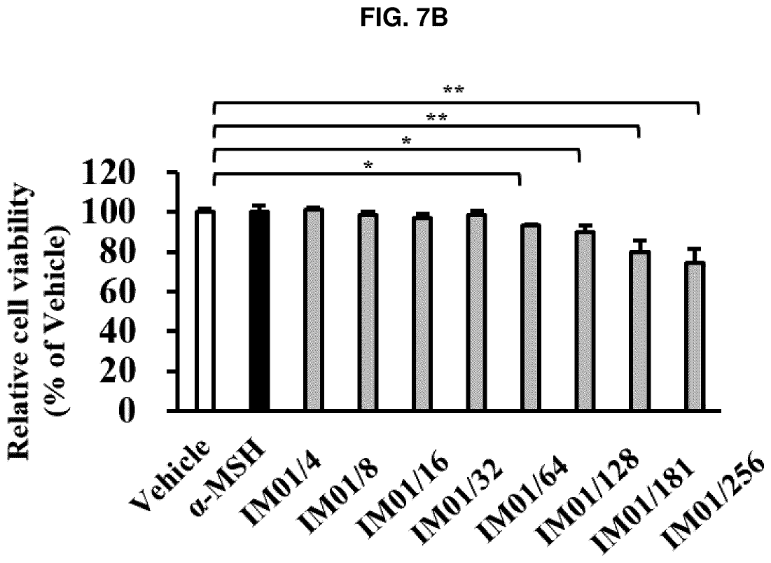

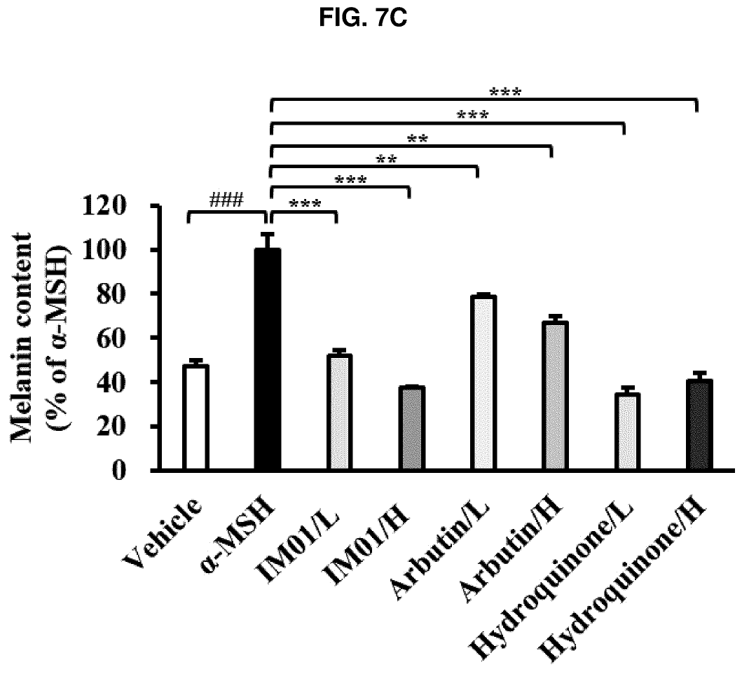

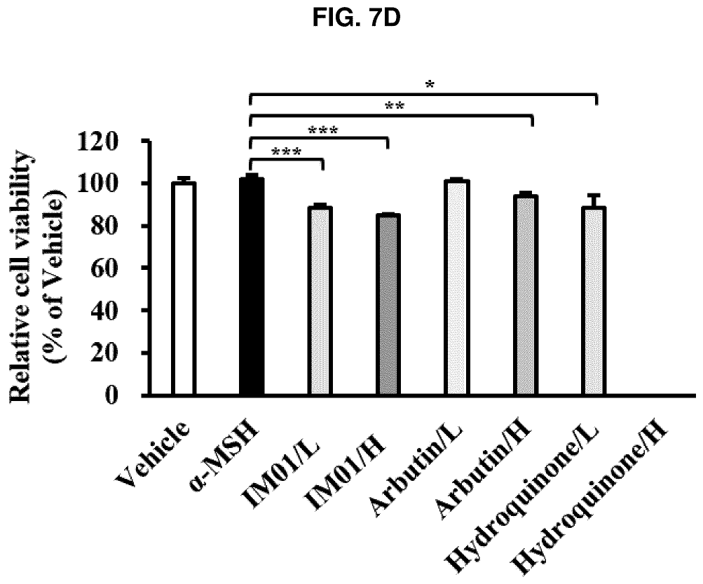

IM01 exhibited whitening efficiency as indicated by the reduction of melanin levels included by α-MSH and the reduction activity is dose-dependent. FIG. 7A. It is determined in this study that the EC₅₀ value of IM01 is around 41.2 μM. No significant toxicity was observed in cells treated with IM01. FIG. 7B. The efficacy and toxicity results are also summarized in Table 24 below.

TABLE 24
In Vitro Efficacy and Toxicity of 4-[(E)-2-(3,5-dihydroxyphenyl)ethenyl]
benzene-1,3-diol

| | | α- | IM01/ | IM01/ | IM01/ | IM01/ | IM01/ | IM01/ | IM01/ | IM01/ |
|---|---|---|---|---|---|---|---|---|---|---|
| Group | Vehicle | MSH | 4 | 8 | 16 | 32 | 64 | 128 | 181 | 256 |
| Relative | 100.0 ± 1.8 | 100.3 ± 2.9 | 101.3 ± 1.2 | 98.4 ± 2.0 | 96.7 ± 2.4 | 98.7 ± 2.2 | 93.0 ± 1.0 | 89.8 ± 3.7 | 79.9 ± 5.6 | 74.6 ± 7.0 |
| cell | | | | | | | | | | |
| viability | | | | | | | | | | |
| (%) | | | | | | | | | | |
| Melanin | 50.9 ± 0.6 | 100.0 ± 7.2 | 90.2 ± 6.1 | 83.2 ± 6.9 | 81.8 ± 4.5 | 67.7 ± 7.1 | 43.0 ± 4.3 | 36.1 ± 2.9 | 34.8 ±3.5 | 25.9 ± 1.5 |
| content | | | | | | | | | | |
| (%) | | | | | | | | | | |

B. In Vitro Efficacy and Toxicity of 4-[(E)-2-(3,5-dihydroxyphenyl)ethenyl]benzene-1,3-diol in Comparison with Other Compounds

Table 25 below lists the study design for evaluating EC50 values of IM01, which contains 4-[(E)-2-(3,5-dihydroxyphenyl)ethenyl]benzene-1,3-diol.

TABLE 25
Study Design of In Vitro Efficacy and Toxicity Studies
Study Design

| | | α- | TA Conc. | | Test | |
|---|---|---|---|---|---|---|
| Study Group | Vehicle | MSH | (μM) | Treatment Time | System | Assay |
| Vehicle | 0.2% | − | − | α-MSH treat 24 hr | B16-F10 | 1. Relative cell |
| | DMSO | | | + | cell line | viability |
| α-MSH | 0.2% | + | − | TA treat 24 hr | | 2. Melanin content |
| | DMSO | | | | | |
| IM01/L | 0.2% | + | 60 | | | |
| | DMSO | | | | | |
| IM01/H | 0.2% | + | 120 | | | |
| | DMSO | | | | | |
| Arbutin/L | 0.2% | + | 450 | | | |
| | DMSO | | | | | |
| Arbutin/H | 0.2% | + | 900 | | | |
| | DMSO | | | | | |
| Hydroquinone/L | DDW | + | 60 | | | |
| Hydroquinone/H | DDW | + | 120 | | | |

Relative to the other compounds investigated in this study, IM01 significantly inhibited melanin production and exhibited low cytotoxicity. FIGS. 7C and 7D. For example, the melanin inhibition activity of IM01 is much greater than arbutin. Hydroquinone (HQ) showed high cytotoxicity. See also Table 26 below.

TABLE 26
In Vitro Efficacy and Toxicity of 4-[(E)-2-(3,5-dihydroxyphenyl)ethenyl]
benzene-1,3-diol and Other Compounds

| | | α- | IM01/ | IM01/ | Arbutin/ | Arbutin/ | | |
|---|---|---|---|---|---|---|---|---|
| Group | Vehicle | MSH | L | H | L | H | HQ/L | HQ/H |
| Relative | 100.0 ± 2.6 | 102.0 ± 1.8 | 88.5 ± 1.4 | 84.9 ± 0.4 | 100.8 ± 1.2 | 93.7 ± 1.6 | 88.2 ± 6.0 | N.D. |
| cell | | | | | | | | |
| viability | | | | | | | | |
| (%) | | | | | | | | |
| Melanin | 47.0 ± 3.0 | 100.0 ± 7.1 | 51.7 ± 2.6 | 37.7 ± 0.4 | 78.5 ± 1.1 | 67.0 ± 2.8 | 34.6 ± 2.8 | 40.5 ± 3.7 |
| content | | | | | | | | |
| (%) | | | | | | | | |

### Example 8. In Vitro Reduction of Hyperpigmentation Activity of Additional Derivatives Design of In Vitro Efficacy Test

B16-F10 cells are a melanoma cell line derived from C₅₇BL/6 mice, which produce melanin and display metastatic behaviors. Thus, B16-F10 cells can be used to study melanogenesis and depigmentation.

Alpha-melanocyte-stimulating hormone (α-MSH) is a peptide hormone capable of inducing melanin production. In this study, the effects of 4-[(E)-2-(3,5-dihydroxyphenyl)ethenyl]benzene-1,3-diol and derivatives samples on inhibition of melanin production were investigated. The experimental design is listed in Table 27.

TABLE 27
Experimental Design of the In Vitro Efficacy Test

| | | | | TA | |
|---|---|---|---|---|---|
| | | | α-MSH | Conc. | Treatment |
| No. | Group | Vehicle | (0.1 μM) | (μM) | Time |
| 1 | Vehicle | 0.2% | − | − | α-MSH treat |
| 2 | α-MSH | DMSO | + | − | 24 hr + |
| 3 | IM01 | | + | + | TA treat 24 hr |
| 4 | IM02 | | + | + | |
| 5 | IM03 | | + | + | |
| 6 | IM04 | | + | + | |
| 7 | IM05 | | + | + | |

IM01: 4-[(E)-2-(3,5-dihydroxyphenyl)ethenyl]benzene-1,3-diol
IM02: 1,2,3-trimethoxy-5-[(Z)-2-phenylethenyl]benzene
IM03: 1-[(E)-2-(2,4-dimethoxyphenyl)ethenyl]-3,5-dimethoxybenzene
IM04: [4-[(E)-2-(3,5-diacetyloxyphenyl)ethenyl]phenyl] acetate
IM05: 5-[(E)-2-(3-hydroxy-4-methoxyphenyl)ethenyl]benzene-1,3-diol

### Methods and Materials

### A. Test Conditions

B16-F10 cells were seeded in a 96-well plate at a density of 1×10⁵ cells/well and cultured for 24 hours. The cells were then incubated with 0.1 μM α-MSH for 24 hours to include melanin production. The 4-[(E)-2-(3,5-dihydroxyphenyl)ethenyl]benzene-1,3-diol and derivatives samples were then added to the cultured cells, which were cultured for an additional 24 hours. No α-MSH was used for the vehicle control group.

### B. Determination of Melanin Content in Each Group

After the cells were washed once with 1 mL of 1×PBS, add 300 μL of 1×Trypsin-EDTA, they were placed in a 37° C. incubator for 5 minutes. Finally, 1 mL of DMEM was added to the cells, and the number of cells were counted by Trypan blue staining.

Cell suspension was centrifuged at 1500 g for 5 minutes at room temperature, followed by supernatant removal and air-dry of the pellet for 15 minutes. 1 N NaOH (containing 10% DMSO) was added to break up the pellet, followed by shaking by ultrasonic waves for 30 minutes and heating in a water bath at 80° C. for 1.5 hours. After cooling down, the suspension was taken and measured for absorbance at 405 nm. Results were calculated by deducting the reagent blank from the absorbance value at 405 nm and divided by the average absorbance value of the α-MSH Group.

### C. Expected Results

It is expected that the 4-[(E)-2-(3,5-dihydroxyphenyl)ethenyl]benzene-1,3-diol and 4-[(E)-2-(3,5-dihydroxyphenyl)ethenyl]benzene-1,3-diol derivatives samples tested herein would significantly reduce melanin content as compared to the control group (with α-MSH only). IM01, IM02, IM03, IM04 and IM05 groups are also expected to reduce melanin production to levels lower than that of the vehicle group (without α-MSH).

It is expected that the 4-[(E)-2-(3,5-dihydroxyphenyl)ethenyl]benzene-1,3-diol and -[(E)-2-(3,5-dihydroxyphenyl)ethenyl]benzene-1,3-diol derivatives may suppressed melanin production induced by α-MSH and reduce baseline melanin production.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for reducing hyperpigmentation in a subject, comprising administering an effective amount of a pharmaceutical composition to a skin site of a subject in need thereof, wherein the pharmaceutical composition comprises a pharmaceutically acceptable carrier and a compound of Formula (I) or a pharmaceutically acceptable salt thereof: 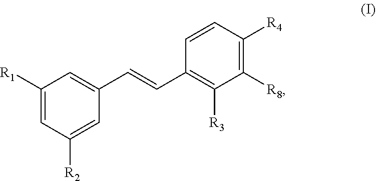
wherein each of R₁, R₂, R₃, R₄, and R₈ independently, is H, halogen, thiol, hydroxyl, C1-4 alkyl, C2-4 alkenyl, OR₅, COOR₆, or —OC(═O)R₇; and
wherein each of R₅, R₆, and R₇ are independently H, or C1-4 alkyl.

2. The method of claim 1, wherein the compound of Formula (I) has following structure: 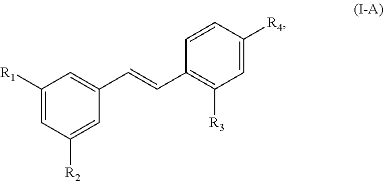
wherein each of R₁, R₂, R₃, and R₄ is set forth in claim 1.

3. The method of claim 1, wherein at least one of R₁, R₂, R₃, and R₄ is hydroxyl or methoxy.

4. The method of claim 3, wherein at least three of R₁, R₂, R₃, and R₄ are hydroxyl or methoxy.

5. The method of claim 1, wherein at least one of R₁, R₂, R₃, and R₄ is OC(═O)R₇, in which R₇ is C1-4 alkyl, optionally methyl.

6. The method of claim 5, wherein at least three of R₁, R₂, R₃, and R₄ are OC(═O)R₇, in which R₇ is C1-4 alkyl, optionally methyl.

7. The method of claim 1, wherein R₈ is hydroxyl.

8. The method of claim 1, wherein the compound has one of the following structures:

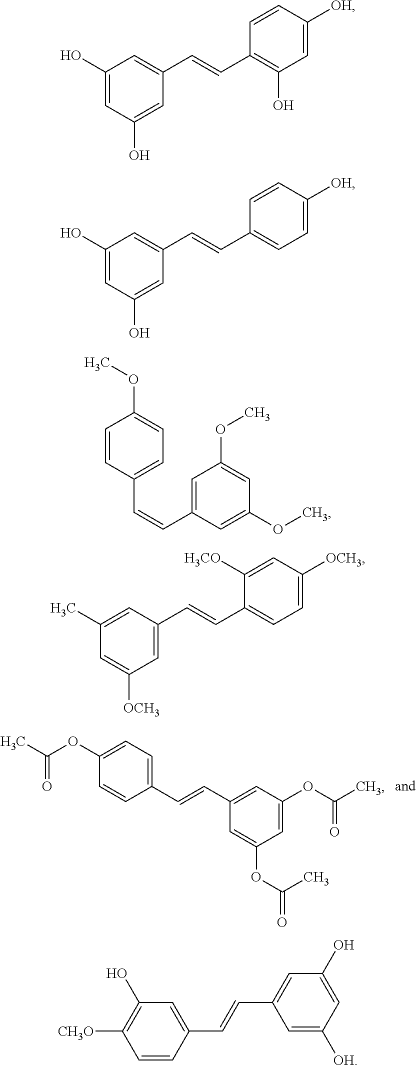

9. The method of claim 1, wherein the pharmaceutically acceptable carrier comprises a non-ionic surfactant having a hydrophilic-lipophilic balance value (HLB value) greater than 5, a solvent, a buffer agent, an oil phase, or a combination thereof.

10. The method of claim 9, wherein the non-ionic surfactant comprises polyoxyl castor oil, and/or polyoxyl hydrogenated castor oil.

11. The method of claim 9, wherein the pharmaceutically acceptable carrier comprises the solvent, which is a polyol or a polymer thereof, acetone, dimethyl sulfoxide, glycerol, alcohol, or a combination thereof, optionally wherein the polyol or polymer thereof is ethylene glycol, polyoxyethylene, polyethylene glycol, or propylene glycol.

12. The method of claim 9, wherein the pharmaceutically acceptable carrier comprises the oil phase, which comprises soybean oil and/or medium-chain triglycerides.

13. The method of claim 1, wherein the pharmaceutical composition comprises a plurality of nanoparticles formed by the pharmaceutically acceptable carrier and the compound of Formula (I), or a pharmaceutically acceptable salt thereof.

14. The method of claim 13, wherein the nanoparticles have an average particle size of up to 200 nm and a polydispersity index (PDI) value of up to 0.4.

15. The method of claim 13, wherein the pharmaceutically acceptable carrier comprises the non-ionic surfactant and wherein the weight ratio between the compound of Formula (I) or the pharmaceutically acceptable salt thereof to the non-ionic surfactant ranges from 1:1 to 1:1000, optionally 1:1 to 1:500.

16. The method of claim 1, wherein the pharmaceutical composition is administered to the skin site via intradermal injection or topical application.

17. The method of claim 1, wherein the pharmaceutical composition further comprises an active agent for reducing hyperpigmentation, wherein the active agent is different from the compound or the pharmaceutically acceptable salt thereof.

18. The method of claim 1, wherein the method further comprises administering to the subject an active agent for reducing hyperpigmentation, wherein the active agent is different from the compound or the pharmaceutically acceptable salt thereof.

19. The method of claim 1, wherein the pharmaceutical composition further comprises an antioxidant.

20. The method of claim 1, wherein the pharmaceutical composition is free of water and/or an antioxidant.
